(19) United States
(12) Patent Application Publication
(10) Pub. No.: US 2014/0175336 A1
(43) Pub. Date: Jun. 26, 2014

(54) CO2 CAPTURE PROCESSES USING ROTARY WHEEL CONFIGURATIONS

(71) Applicants: Ramesh Gupta, Berkley Heights, NJ (US); Hans Thomann, Bedminster, NJ (US); Richard D. Lenz, Tonawanda, NY (US); Hugo S. Caram, Allentown, PA (US)

(72) Inventors: Ramesh Gupta, Berkley Heights, NJ (US); Hans Thomann, Bedminster, NJ (US); Richard D. Lenz, Tonawanda, NY (US); Hugo S. Caram, Allentown, PA (US)

(73) Assignee: EXXONMOBIL RESEARCH AND ENGINEERING COMPANY, Annandale, NJ (US)

(21) Appl. No.: 14/085,954

(22) Filed: Nov. 21, 2013

Related U.S. Application Data
(60) Provisional application No. 61/740,025, filed on Dec. 20, 2012.

(51) Int. Cl. B01D 53/14; C07C 7/12

(52) U.S. Cl. CPC B01D 53/1475; C07C 7/12
USPC 252/373; 423/230; 585/824

(57) ABSTRACT

The disclosure relates to a continuous or semi-continuous, cyclic, countercurrent sorption-desorption method for enhanced control, separation, and/or purification of CO₂ from one or more sources of a mixture of gases (and/or carbonaceous liquids that have sufficient vapor pressure) through integrated use of solid monolithic sorbents having a selectivity for sorption of the CO₂.

### CROSS-REFERENCE TO RELATED APPLICATIONS

This application claims the benefit of U.S. Application Ser. No. 61/740,025, filed on Dec. 20, 2012; which is incorporated by reference herein in its entirety.

### FIELD OF THE INVENTION

This invention relates to methods for enhanced control, separation, and/or purification of CO₂ from one or more sources of a mixture of gases in a continuous or semi-continuous, cyclic sorption-desorption process.

### BACKGROUND OF THE INVENTION

Global climate change concerns may necessitate capture of CO₂, e.g., from flue gases and other process streams. One traditional approach involves absorption of CO₂ with an amine solution, such as monoethanolamine (MEA), other ethanolamines, or certain amine mixtures, which solution is then thermally regenerated and recycled. This traditional approach is capital and energy intensive. There is considerable prior art in this area of conventional liquid sorption.

There is also some level of prior art regarding solid sorbents and rotary wheels. For example, there are the following scholarly articles: C. Y. Pan et al., Chemical Engineering Science, 22 (1967), 285; C. Y. Pan et al., Chemical Engineering Science, 25 (1970), 1653; Ralph T. Yang, Gas Separation and Adsorption Processes, Imperial College Press, 1997; Y. Matsukuma et al., “Study of CO₂ recovery system from flue gas by honeycomb type adsorbent I.”, Kagaku Kogaku Ronbunshu, 32(2), 2006, 138-145; Y. Matsukuma et al., “Simulation of CO₂ recovery system from flue gas by honeycomb type adsorbent: II. Optimization of CO₂ recovery system and proposal for actual plant”, Kagaku Kogaku Ronbunshu, 32(2), 2006, 146; C. Shen et al., “Adsorption Equilibria and Kinetics of CO₂ and N₂ on activated Carbon Beads”, Chemical Engineering Science, 160 (2010), 398-407; Z. C. Liu et al., Carbon, 37 (4), 1999, 663-667; and G. Krishnan, “Development of Novel Carbon Sorbents for CO₂ Capture”, presented at the 2010 NETL CO₂ Capture Technology Meeting, 13-17 Sep. 2010, Pittsburgh, Pa. There are also the following patent-related publications: U.S. Patent Application Publication Nos. 2005/0215481, 2005/0217481, and 2009/0214902; U.S. Pat. Nos. 4,778,492, 6,500,236, 6,596,248, 6,521,026, 6,783,738, 7,022,168, and 7,166,149; European Patent Nos. EP 1138369 and EP 2258879; Japanese Patent Publication No. 2003181242; and Japanese Patent No. 4414110.

It would be highly desirable to employ a sorption method that is less capital and energy intensive than conventional liquid amine sorbents and that can provide an efficiency advantage.

### SUMMARY OF THE INVENTION

One aspect of the invention relates to a continuous or semi-continuous, cyclic, countercurrent sorption-desorption method for enhanced control, separation, and/or purification of CO₂ from one or more sources of a mixture of gases (and/or carbonaceous liquids that have sufficient vapor pressure) through integrated use of solid monolithic sorbents having a selectivity for sorption of the CO₂. Though described herein as “monolithic”, the solid sorbents according to the invention can be aggregated particulate, monolithic, and/or structured, so long as they behave as if solid and cohesive from the point of view of the contact with the gaseous/fluid streams described herein. Although liquid amine-based materials can be considered conventional sorbents, solid monolithic sorbents (particularly when employed in a rotating wheel-type configuration) can have distinct advantages over conventional sorbents, including, but not necessarily limited to, the ability to process relatively large gas volumes/flow rates, continuous operation, and few/no valves (thus little or no flow switching required).

Typical flue gas volumes of about 50-100 million ft³/hr can be emitted from a large refinery or a coal power plant, and, as such, the methods according to the invention can advantageously have adequate adsorption capacity to capture the CO₂ content, which can be realized, e.g., by using at least 2 to about 10 large rotary wheels that may each have, in one embodiment, diameters of approximately 10-80 feet and widths of approximately 6 inches to 2 feet, or more. Additionally or alternately, the gas velocity entering such rotary adsorbent wheels can be up to about 15 ft/sec or more, and/or the pressure drop across such rotary adsorbent wheels can be less than 4 psi, e.g., less than 3 psi, less than 2 psi, less than 1 psi, less than 0.5 psi, less than 0.3 psi, less than 0.2 psi, or less than 0.1 psi.

In order for solid adsorbent to be generally effective for CO₂ capture, at least one, and preferably most or all, of the following can advantageously apply: the sorbent material can have a relatively high sorption capacity for CO₂, so as to reduce/minimize the required adsorbent volume and/or process footprint; the sorbent material can have relatively fast CO₂ sorption and desorption kinetics, e.g., so that relatively short sorption-desorption cycle times (e.g., about 15 seconds to about 10 minutes) can be utilized, allowing increased/optimized productivity for a given size plant; the sorbent material can have a relatively high tolerance to water, e.g., so that moisture in the flue gas does not significantly reduce CO₂ sorption; the sorbent material can have an acceptable tolerance to contaminants (in flue gases, those can include SOₓ and/or NOₓ), with no significant reduction in CO₂ capacity, e.g., due to irreversible binding or chemical reaction of such contaminants with the sorption sites; the sorbent material can have relative stability to temperature cycling and steam; and the sorbent material can have relatively high CO₂/N₂ sorption selectivity (flue gas can typically exhibit as high as 85-90% N₂ content and generally about 20% or less CO₂ content).

In situations where the solid monolithic sorbent(s) is(are) comprised of alkali modified (basic) alumina, one advantage can be that they can adsorb unusually high quantities of CO₂ at temperatures above 100° C., which can allow a lower temperature differential between the adsorption and desorption steps/stages. Such an arrangement can offer much lower energy requirements, higher achievable CO₂ purities, faster cycle times, and thus typically smaller hardware than other thermal swing adsorption (TSA) processes and/or other processes operating at less than 100° C. Other advantages of utilizing alkali modified (basic) alumina sorbent materials in the methods according to the invention can include, but are not necessarily limited to, relatively low heat of sorption, relative to other adsorbents, which can result in relatively low energy requirements for desorption and/or regeneration steps; and relatively fast adsorption and desorption kinetics allowing a shorter sorption-desorption cycle time, which can manifest as higher throughput for a given size adsorption system or as relatively smaller footprint for a given throughput. Additionally or alternately in situations where the solid monolithic sorbent(s) is(are) comprised of alkali modified (basic) alumina, the modified alumina may optionally be disposed as a wash-coat on the surface(s) of the solid monolithic sorbent(s).

Wash-coats can be used to introduce functionality to a solid monolithic sorbent and/or to augment already existing functionality. For example, though it is possible to use multiple, separate solid monolithic sorbents (e.g., a first upstream sorbent to remove water from a flue gas, followed by a second downstream sorbent for CO₂ removal), wash-coating can be used to combine such processes into a single, layered solid monolithic sorbent. When a wash-coat is utilized, its thickness can be tailored (optimized) to allow rapid CO₂-adsorbent mass exchange and to advantageously facilitate a large capacity for adsorbed CO₂. In general, a thicker wash-coat can increase sorbent capacity, but diffusion resistance can often limit the rate at which the CO₂ can be adsorbed/desorbed. Alternately, a relatively thin wash-coat can allow relatively rapid CO₂ exchange but with attendant lower sorbent capacity increase, if any is appreciable.

Another variable in cyclic sorption-desorption methods can include pressure drop across each solid monolithic sorbent used. For instance, where flue gas is a/the source of mixed gas, each solid monolithic sorbent can be designed to exhibit a relatively low pressure drop (e.g., less than 4 psi, less than 3 psi, less than 2 psi, less than 1 psi, less than 0.5 psi, less than 0.3 psi, less than 0.2 psi, or less than 0.1 psi). This can be critical, since absolute flue gas pressures can tend to be near ambient pressures. Though it is possible to boost flue gas pressure using a compressor and/or a blower fan, this can generally be unattractive for economic reasons. Narrow monolith flow channels can allow a larger CO₂-sorbent contact area and can be desirable, in some embodiments, from mass transfer considerations. However, narrower flow channels can undesirably increase pressure drop. The size of the channels can be tailored/optimized to achieve acceptable contact area within the constraint of permissible pressure drop. The channels can be circular or of any other shape (such as rectangular, hexagonal, or the like, or modifications thereof, e.g., to include protrusions into the channel for additional contact area) consistent with the requirements of acceptably high mass transfer area and acceptably low pressure drop.

Adsorption can tend to lead to the generation of heat. Rising temperature in the solid monolithic sorbent(s) can tend to reduce sorption capacity. The integration of additional cooling mechanisms to combat adiabatic temperature increases can be effected, e.g., by injection of atomized liquid droplets or sprays or running liquid streams through the sorbent bed or monolith channels. This liquid can advantageously serve to remove heat by vaporization and/or through sensible cooling. Strategies for a cooling step prior to CO₂ sorption can include, but are not limited to, at least partial cooling using air blowers, for example. Integrated cooling schemes can involve air/water droplets (e.g., created by atomizers/sprays), which could achieve increased heat management via evaporative cooling mechanisms. Unsaturated and/or relatively dry (e.g., less than 50% relative humidity) air could additionally or alternately help dehumidify the sorbent beds.

### BRIEF DESCRIPTION OF THE DRAWINGS

FIG. 1 shows an example of a single rotary sorption monolithic wheel in which the sorption is achieved in an overall countercurrent manner, with respect to the rotation of the wheel.

FIG. 2 shows a schematic of a counter-rotating two wheel system operated in a countercurrent manner, using a method according to the invention.

FIG. 3 shows a schematic of a counter-rotating two wheel system operated in a countercurrent manner, using a method according to the invention similar to FIG. 2, except with the added concepts of separate water spray (evaporative cooling) steps followed by additional drying/cooling steps.

FIG. 4 shows a schematic of a counter-rotating two wheel system operated using a method according to the invention containing multiple sorption and multiple desorption steps, in a countercurrent manner, but where one wheel is isolated from the other wheel in its flow arrangements.

FIG. 5 shows a schematic of a counter-rotating two wheel system operated using a method according to the invention containing multiple sorption and multiple desorption steps, in a countercurrent manner, but where the flow arrangements for both wheels are interconnected.

FIG. 6 shows a stepwise diagram of the flow arrangement of the schematic shown in FIG. 5.

### DETAILED DESCRIPTION OF THE EMBODIMENTS

The present invention can involve a method for enhanced control, separation, and/or purification of CO₂ from one or more sources of a mixture of gases (and/or carbonaceous liquids that have sufficient vapor pressure). Although the present invention is described with reference to CO₂, it should be understood that such methods/systems described herein can additionally or alternately be used to control, separate, and/or purify other gases, individually and/or collectively; such other gases can optionally include, but are not limited to, light (e.g., C₁-C₄ or C₁-C₃) hydrocarbons (i.e., saturated, such as methane, ethane, propane, n-butane, isobutane, and the like, and combinations thereof, and/or unsaturated, such as ethylene, propylene, 1-butene, 2-butenes, isobutylene, butadiene, and the like, and combinations thereof), water, hydrogen sulfide, carbon monoxide, carbonyl sulfide, SOₓ, NOₓ, and the like, and combinations thereof.

Advantageously, at least two solid monolithic sorbents can be provided being the same or different from each other (preferably, but not necessarily, the same), the sorbents each having a selectivity for CO₂ in a continuous or semi-continuous, cyclic, countercurrent sorption-desorption process. Although the sorbent materials are referred to herein as solid and monolithic, they need only act or behave as solid and monolithic with respect to the flow of the mixed gas source(s). For instance, they can alternately comprise (optionally packed) granular particulate sorbent materials and/or inert (structured) packing onto which sorbent functionality (e.g., amine functionality or the like) can be immobilized/grafted. In certain embodiments, the at least two solid monolithic sorbents can be oriented such that their cross-sectional planes are approximately parallel and such that they rotate about a common rotational axis, e.g., that is substantially perpendicular to the cross-sectional planes of the monolithic sorbents. In such embodiments, each successive solid monolithic sorbent can have a counter-rotational direction that alternates between clockwise and counterclockwise, as viewed along the common rotational axis.

When the term “selectivity” is used herein with respect to the propensity of a sorbent to favor sorption of a desired component (in this case, typically CO₂) over an undesired component, it should be understood that such “selectivity” is based on approximately an equilibrium sorption process with the sorbent, and not on a kinetic sorption process. That means that selectivities described herein represent competitive sorption between desired and undesired components on a time scale that is long enough to approximate equilibrium—whether such a sufficiently long time scale may be on the order of portions of seconds or multiple hours (or anywhere in between) should not be particularly relevant. At times herein, selectivity can be expressed only with respect to the desired component (e.g., CO₂), leaving the undesired component unnamed, merely to express the importance of the desired component to the separation.

The source(s) of mixed gas can advantageously (collectively and/or each) comprise from about 1 vol % to about 70 vol % CO₂, e.g., from about 1 vol % to about 60 vol % CO₂, from about 1 vol % to less than 50 vol % CO₂, from about 1 vol % to about 45 vol % CO₂, from about 1 vol % to about 40 vol % CO₂, from about 1 vol % to about 30 vol % CO₂, from about 1 vol % to about 25 vol % CO₂, from about 1 vol % to about 20 vol % CO₂, from about 1 vol % to about 15 vol % CO₂, from about 1 vol % to about 10 vol % CO₂, from about 1 vol % to about 5 vol % CO₂, from about 5 vol % to about 70 vol % CO₂, from about 5 vol % to about 60 vol % CO₂, from about 5 vol % to less than 50 vol % CO₂, from about 5 vol % to about 45 vol % CO₂, from about 5 vol % to about 40 vol % CO₂, from about 5 vol % to about 30 vol % CO₂, from about 5 vol % to about 25 vol % CO₂, from about 5 vol % to about 20 vol % CO₂, from about 5 vol % to about 15 vol % CO₂, from about 5 vol % to about 10 vol % CO₂, from about 10 vol % to about 70 vol % CO₂, from about 10 vol % to about 60 vol % CO₂, from about 10 vol % to less than 50 vol % CO₂, from about 10 vol % to about 45 vol % CO₂, from about 10 vol % to about 40 vol % CO₂, from about 10 vol % to about 30 vol % CO₂, from about 10 vol % to about 25 vol % CO₂, from about 10 vol % to about 20 vol % CO₂, from about 10 vol % to about 15 vol % CO₂, from about 15 vol % to about 70 vol % CO₂, from about 15 vol % to about 60 vol % CO₂, from about 15 vol % to less than 50 vol % CO₂, from about 15 vol % to about 45 vol % CO₂, from about 15 vol % to about 40 vol % CO₂, from about 15 vol % to about 30 vol % CO₂, from about 15 vol % to about 25 vol % CO₂, from about 15 vol % to about 20 vol % CO₂, from about 20 vol % to about 70 vol % CO₂, from about 20 vol % to about 60 vol % CO₂, from about 20 vol % to less than 50 vol % CO₂, from about 20 vol % to about 45 vol % CO₂, from about 20 vol % to about 40 vol % CO₂, from about 20 vol % to about 30 vol % CO₂, from about 20 vol % to about 25 vol % CO₂, from about 25 vol % to about 70 vol % CO₂, from about 25 vol % to about 60 vol % CO₂, from about 25 vol % to less than 50 vol % CO₂, from about 25 vol % to about 45 vol % CO₂, from about 25 vol % to about 40 vol % CO₂, from about 25 vol % to about 30 vol % CO₂, from about 30 vol % to about 70 vol % CO₂, from about 30 vol % to about 60 vol % CO₂, from about 30 vol % to less than 50 vol % CO₂, from about 30 vol % to about 45 vol % CO₂, from about 30 vol % to about 40 vol % CO₂, from about 40 vol % to about 70 vol % CO₂, from about 40 vol % to about 60 vol % CO₂, from about 40 vol % to less than 50 vol % CO₂, from about 40 vol % to about 45 vol % CO₂, or from about 50 vol % to about 70 vol % CO₂.

Additionally or alternately, the source(s) of mixed gas can (collectively and/or each) comprise from about 0.1 vol % to about 40 vol % moisture, e.g., from about 0.1 vol % to about 35 vol % moisture, from about 0.1 vol % to about 30 vol % moisture, from about 0.1 vol % to about 25 vol % moisture, from about 0.1 vol % to about 20 vol % moisture, from about 0.1 vol % to about 15 vol % moisture, from about 0.1 vol % to about 10 vol % moisture, from about 0.1 vol % to about 5 vol % moisture, from about 0.1 vol % to about 3 vol % moisture, from about 0.1 vol % to about 1 vol % moisture, from about 0.3 vol % to about 40 vol % moisture, from about 0.3 vol % to about 35 vol % moisture, from about 0.3 vol % to about 30 vol % moisture, from about 0.3 vol % to about 25 vol % moisture, from about 0.3 vol % to about 20 vol % moisture, from about 0.3 vol % to about 15 vol % moisture, from about 0.3 vol % to about 10 vol % moisture, from about 0.3 vol % to about 5 vol % moisture, from about 0.3 vol % to about 3 vol % moisture, from about 0.3 vol % to about 1 vol % moisture, from about 0.5 vol % to about 40 vol % moisture, from about 0.5 vol % to about 35 vol % moisture, from about 0.5 vol % to about 30 vol % moisture, from about 0.5 vol % to about 25 vol % moisture, from about 0.5 vol % to about 20 vol % moisture, from about 0.5 vol % to about 15 vol % moisture, from about 0.5 vol % to about 10 vol % moisture, from about 0.5 vol % to about 5 vol % moisture, from about 0.5 vol % to about 3 vol % moisture, from about 0.5 vol % to about 1 vol % moisture, from about 1 vol % to about 40 vol % moisture, from about 1 vol % to about 35 vol % moisture, from about 1 vol % to about 30 vol % moisture, from about 1 vol % to about 25 vol % moisture, from about 1 vol % to about 20 vol % moisture, from about 1 vol % to about 15 vol % moisture, from about 1 vol % to about 10 vol % moisture, from about 1 vol % to about 5 vol % moisture, from about 5 vol % to about 40 vol % moisture, from about 5 vol % to about 35 vol % moisture, from about 5 vol % to about 30 vol % moisture, from about 5 vol % to about 25 vol % moisture, from about 5 vol % to about 20 vol % moisture, from about 5 vol % to about 15 vol % moisture, from about 5 vol % to about 10 vol % moisture, from about 10 vol % to about 40 vol % moisture, from about 10 vol % to about 35 vol % moisture, from about 10 vol % to about 30 vol % moisture, from about 10 vol % to about 25 vol % moisture, from about 10 vol % to about 20 vol % moisture, from about 10 vol % to about 15 vol % moisture, from about 20 vol % to about 40 vol % moisture, from about 20 vol % to about 35 vol % moisture, from about 20 vol % to about 30 vol % moisture, or from about 30 vol % to about 40 vol % moisture.

Alternately, in embodiments where one or more of the at least two solid monolithic sorbents are especially sensitive to the presence of moisture (e.g., where moisture substantially shortens sorbent useful life, substantially reduces sorbent activity, substantially reduces sorbent selectivity for the target gas component(s), substantially detrimentally affects sorbent structural and/or chemical stability, or the like, or a combination thereof), the moisture content of the source(s) of mixed gas can (collectively and/or each) be, or can be pre-treated to be, about 200 vppm or less, e.g., about 100 vppm or less, about 75 vppm or less, about 50 vppm or less, about 25 vppm or less, about 10 vppm or less, about 5 vppm or less, about 3 vppm or less, about 1 vppm or less, about 500 vppb or less, or about 250 vppb or less. In such alternate moisture-sensitive embodiments, though there may not necessarily be a lower limit on moisture content, it can be practically very difficult to achieve (and/or to experimentally measure) moisture contents below about 10 vppb. Additionally or alternately in such alternate moisture-sensitive embodiments, the source(s) of mixed gas can have, or can be treated to have, a dew point of about −10° C. or less, e.g., about −15° C. or less, about −20° C. or less, about −25° C. or less, about −30° C. or less, about −35° C. or less, about −40° C. or less, about −45° C. or less, or about −50° C. or less; in such embodiments, though there may not necessarily be a lower limit on dew points, it can be practically very difficult to achieve a dew point below about −100° C. A non-limiting example of a moisture-sensitive sorbent material can include 13X molecular sieve, and potentially other high alumina content zeolites.

Further additionally or alternately, the source(s) of mixed gas can (collectively and/or each) comprise at least about 1 vol % C₁-C₃ hydrocarbons, e.g., at least about 3 vol % C₁-C₃ hydrocarbons, at least about 5 vol % C₁-C₃ hydrocarbons, at least about 10 vol % C₁-C₃ hydrocarbons, at least about 15 vol % C₁-C₃ hydrocarbons, at least about 20 vol % C₁-C₃ hydrocarbons, at least about 25 vol % C₁-C₃ hydrocarbons, at least about 30 vol % C₁-C₃ hydrocarbons, at least about 35 vol % C₁-C₃ hydrocarbons, at least about 40 vol % C₁-C₃ hydrocarbons, at least about 45 vol % C₁-C₃ hydrocarbons, at least about 50 vol % C₁-C₃ hydrocarbons, at least about 55 vol % C₁-C₃ hydrocarbons, at least about 60 vol % C₁-C₃ hydrocarbons, at least about 65 vol % C₁-C₃ hydrocarbons, at least about 70 vol % C₁-C₃ hydrocarbons, or at least about 75 vol % C₁-C₃ hydrocarbons. Still further additionally or alternately, the source(s) of mixed gas can (collectively and/or each) comprise up to about 99.9 vol % C₁-C₃ hydrocarbons, e.g., up to about 99.5 vol % C₁-C₃ hydrocarbons, up to about 99 vol % C₁-C₃ hydrocarbons, up to about 98 vol % C₁-C₃ hydrocarbons, up to about 97 vol % C₁-C₃ hydrocarbons, up to about 96 vol % C₁-C₃ hydrocarbons, up to about 95 vol % C₁-C₃ hydrocarbons, up to about 92.5 vol % C₁-C₃ hydrocarbons, up to about 90 vol % C₁-C₃ hydrocarbons, up to about 85 vol % C₁-C₃ hydrocarbons, up to about 80 vol % C₁-C₃ hydrocarbons, up to about 75 vol % C₁-C₃ hydrocarbons, up to about 70 vol % C₁-C₃ hydrocarbons, up to about 65 vol % C₁-C₃ hydrocarbons, up to about 60 vol % C₁-C₃ hydrocarbons, up to about 55 vol % C₁-C₃ hydrocarbons, less than 50 vol % C₁-C₃ hydrocarbons, up to about 45 vol % C₁-C₃ hydrocarbons, up to about 40 vol % C₁-C₃ hydrocarbons, up to about 35 vol % C₁-C₃ hydrocarbons, up to about 30 vol % C₁-C₃ hydrocarbons, up to about 25 vol % C₁-C₃ hydrocarbons, up to about 20 vol % C₁-C₃ hydrocarbons, up to about 15 vol % C₁-C₃ hydrocarbons, up to about 10 vol % C₁-C₃ hydrocarbons, up to about 5.0 vol % C₁-C₃ hydrocarbons, or up to about 1.0 vol % C₁-C₃ hydrocarbons.

Yet further additionally or alternately, the source(s) of mixed gas can (collectively and/or each) comprise from about 3 vppm to about 5000 vppm SOₓ (e.g., from about 3 vppm to about 3000 vppm SOₓ, from about 3 vppm to about 2000 vppm SOₓ, from about 3 vppm to about 1000 vppm SOₓ, from about 3 vppm to about 500 vppm SOₓ, from about 3 vppm to about 300 vppm SOₓ, from about 3 vppm to about 100 vppm SOₓ, from about 3 vppm to about 75 vppm SOₓ, from about 3 vppm to about 50 vppm SOₓ, from about 3 vppm to about 25 vppm SOₓ, from about 3 vppm to about 10 vppm SOₓ, from about 5 vppm to about 5000 vppm SOₓ, from about 5 vppm to about 3000 vppm SOₓ, from about 5 vppm to about 2000 vppm SOₓ, from about 5 vppm to about 1000 vppm SOₓ, from about 5 vppm to about 500 vppm SOₓ, from about 5 vppm to about 300 vppm SOₓ, from about 5 vppm to about 100 vppm SOₓ, from about 5 vppm to about 75 vppm SOₓ, from about 5 vppm to about 50 vppm SOₓ, from about 5 vppm to about 25 vppm SOₓ, from about 5 vppm to about 10 vppm SOₓ, from about 10 vppm to about 5000 vppm SOₓ, from about 10 vppm to about 3000 vppm SOₓ, from about 10 vppm to about 2000 vppm SOₓ, from about 10 vppm to about 1000 vppm SOₓ, from about 10 vppm to about 500 vppm SOₓ, from about 10 vppm to about 300 vppm SOₓ, from about 10 vppm to about 100 vppm SOₓ, from about 10 vppm to about 75 vppm SOₓ, from about 10 vppm to about 50 vppm SOₓ, from about 10 vppm to about 25 vppm SOₓ, from about 25 vppm to about 5000 vppm SOₓ, from about 25 vppm to about 3000 vppm SOₓ, from about 25 vppm to about 2000 vppm SOₓ, from about 25 vppm to about 1000 vppm SOₓ, from about 25 vppm to about 500 vppm SOₓ, from about 25 vppm to about 300 vppm SOₓ, from about 25 vppm to about 100 vppm SOₓ, from about 25 vppm to about 75 vppm SOₓ, from about 25 vppm to about 50 vppm SOₓ, from about 50 vppm to about 5000 vppm SOₓ, from about 50 vppm to about 3000 vppm SOₓ, from about 50 vppm to about 2000 vppm SOₓ, from about 50 vppm to about 1000 vppm SOₓ, from about 50 vppm to about 500 vppm SOₓ, from about 50 vppm to about 300 vppm SOₓ, from about 50 vppm to about 100 vppm SOₓ, from about 100 vppm to about 5000 vppm SOₓ, from about 100 vppm to about 3000 vppm SOₓ, from about 100 vppm to about 2000 vppm SOₓ, from about 100 vppm to about 1000 vppm SOₓ, from about 100 vppm to about 500 vppm SOₓ, from about 500 vppm to about 5000 vppm SOₓ, from about 500 vppm to about 3000 vppm SOₓ, from about 500 vppm to about 2000 vppm SOₓ, or from about 1000 vppm to about 5000 vppm SOₓ), from about 3 vppm to about 5000 vppm NO (e.g., from about 3 vppm to about 3000 vppm NOₓ, from about 3 vppm to about 2000 vppm NOₓ, from about 3 vppm to about 1000 vppm NOₓ, from about 3 vppm to about 500 vppm NOₓ, from about 3 vppm to about 300 vppm NOₓ, from about 3 vppm to about 100 vppm NOₓ, from about 3 vppm to about 75 vppm NOₓ, from about 3 vppm to about 50 vppm NOₓ, from about 3 vppm to about 25 vppm NOₓ, from about 3 vppm to about 10 vppm NOₓ, from about 5 vppm to about 5000 vppm NOₓ, from about 5 vppm to about 3000 vppm NOₓ, from about 5 vppm to about 2000 vppm NOₓ, from about 5 vppm to about 1000 vppm NOₓ, from about 5 vppm to about 500 vppm NOₓ, from about 5 vppm to about 300 vppm NOₓ, from about 5 vppm to about 100 vppm NOₓ, from about 5 vppm to about 75 vppm NOₓ, from about 5 vppm to about 50 vppm NOₓ, from about 5 vppm to about 25 vppm NOₓ, from about 5 vppm to about 10 vppm NOₓ, from about 10 vppm to about 5000 vppm NOₓ, from about 10 vppm to about 3000 vppm NOₓ, from about 10 vppm to about 2000 vppm NOₓ, from about 10 vppm to about 1000 vppm NOₓ, from about 10 vppm to about 500 vppm NOₓ, from about 10 vppm to about 300 vppm NOₓ, from about 10 vppm to about 100 vppm NOₓ, from about 10 vppm to about 75 vppm NOₓ, from about 10 vppm to about 50 vppm NOₓ, from about 10 vppm to about 25 vppm NOₓ, from about 25 vppm to about 5000 vppm NOₓ, from about 25 vppm to about 3000 vppm NOₓ, from about 25 vppm to about 2000 vppm NOₓ, from about 25 vppm to about 1000 vppm NOₓ, from about 25 vppm to about 500 vppm NOₓ, from about 25 vppm to about 300 vppm NOₓ, from about 25 vppm to about 100 vppm NOₓ, from about 25 vppm to about 75 vppm NOₓ, from about 25 vppm to about 50 vppm NOₓ, from about 50 vppm to about 5000 vppm NOₓ, from about 50 vppm to about 3000 vppm NOₓ, from about 50 vppm to about 2000 vppm NOₓ, from about 50 vppm to about 1000 vppm NOₓ, from about 50 vppm to about 500 vppm NOₓ, from about 50 vppm to about 300 vppm NOₓ, from about 50 vppm to about 100 vppm NOₓ, from about 100 vppm to about 5000 vppm NOₓ, from about 100 vppm to about 3000 vppm NOₓ, from about 100 vppm to about 2000 vppm NOₓ, from about 100 vppm to about 1000 vppm NOₓ, from about 100 vppm to about 500 vppm NOₓ, from about 500 vppm to about 5000 vppm NOₓ, from about 500 vppm to about 3000 vppm NOₓ, from about 500 vppm to about 2000 vppm NOₓ, or from about 1000 vppm to about 5000 vppm NOₓ), from about 0.1 vol % to less than 50 vol % H₂ (e.g., from about 0.1 vol % to about 45 vol % H₂, from about 0.1 vol % to about 40 vol % H₂, from about 0.1 vol % to about 35 vol % H₂, from about 0.1 vol % to about 30 vol % H₂, from about 0.1 vol % to about 25 vol % H₂, from about 0.1 vol % to about 20 vol % H₂, from about 0.1 vol % to about 15 vol % H₂, from about 0.1 vol % to about 10 vol % H₂, from about 0.1 vol % to about 5 vol % H₂, from about 0.1 vol % to about 3 vol % H₂, from about 0.1 vol % to about 1 vol % H₂, from about 0.3 vol % to less than 50 vol % H₂, from about 0.3 vol % to about 45 vol % H₂, from about 0.3 vol % to about 40 vol % H₂, from about 0.3 vol % to about 35 vol % H₂, from about 0.3 vol % to about 30 vol % H₂, from about 0.3 vol % to about 25 vol % H₂, from about 0.3 vol % to about 20 vol % H₂, from about 0.3 vol % to about 15 vol % H₂, from about 0.3 vol % to about 10 vol % H₂, from about 0.3 vol % to about 5 vol % H₂, from about 0.3 vol % to about 3 vol % H₂, from about 0.3 vol % to about 1 vol % H₂, from about 0.5 vol % to less than 50 vol % H₂, from about 0.5 vol % to about 45 vol % H₂, from about 0.5 vol % to about 40 vol % H₂, from about 0.5 vol % to about 35 vol % H₂, from about 0.5 vol % to about 30 vol % H₂, from about 0.5 vol % to about 25 vol % H₂, from about 0.5 vol % to about 20 vol % H₂, from about 0.5 vol % to about 15 vol % H₂, from about 0.5 vol % to about 10 vol % H₂, from about 0.5 vol % to about 5 vol % H₂, from about 0.5 vol % to about 3 vol % H₂, from about 0.5 vol % to about 1 vol % H₂, from about 1 vol % to less than 50 vol % H₂, from about 1 vol % to about 45 vol % H₂, from about 1 vol % to about 40 vol % H₂, from about 1 vol % to about 35 vol % H₂, from about 1 vol % to about 30 vol % H₂, from about 1 vol % to about 25 vol % H₂, from about 1 vol % to about 20 vol % H₂, from about 1 vol % to about 15 vol % H₂, from about 1 vol % to about 10 vol % H₂, from about 1 vol % to about 5 vol % H₂, from about 1 vol % to about 3 vol % H₂, from about 3 vol % to less than 50 vol % H₂, from about 3 vol % to about 45 vol % H₂, from about 3 vol % to about 40 vol % H₂, from about 3 vol % to about 35 vol % H₂, from about 3 vol % to about 30 vol % H₂, from about 3 vol % to about 25 vol % H₂, from about 3 vol % to about 20 vol % H₂, from about 3 vol % to about 15 vol % H₂, from about 3 vol % to about 10 vol % H₂, from about 3 vol % to about 5 vol % H₂, from about 5 vol % to less than 50 vol % H₂, from about 5 vol % to about 45 vol % H₂, from about 5 vol % to about 40 vol % H₂, from about 5 vol % to about 35 vol % H₂, from about 5 vol % to about 30 vol % H₂, from about 5 vol % to about 25 vol % H₂, from about 5 vol % to about 20 vol % H₂, from about 5 vol % to about 15 vol % H₂, from about 5 vol % to about 10 vol % H₂, from about 10 vol % to less than 50 vol % H₂, from about 10 vol % to about 45 vol % H₂, from about 10 vol % to about 40 vol % H₂, from about 10 vol % to about 35 vol % H₂, from about 10 vol % to about 30 vol % H₂, from about 10 vol % to about 25 vol % H₂, from about 10 vol % to about 20 vol % H₂, or from about 20 vol % to less than 50 vol % H₂), from about 3 vppm to about 10000 vppm H₂S (e.g., from about 3 vppm to about 7500 vppm H₂S, from about 3 vppm to about 5000 vppm H₂S, from about 3 vppm to about 2500 vppm H₂S, from about 3 vppm to about 1000 vppm H₂S, from about 3 vppm to about 500 vppm H₂S, from about 3 vppm to about 250 vppm H₂S, from about 3 vppm to about 100 vppm H₂S, from about 3 vppm to about 75 vppm H₂S, from about 3 vppm to about 50 vppm H₂S, from about 3 vppm to about 25 vppm H₂S, from about 3 vppm to about 10 vppm H₂S, from about 5 vppm to about 10000 vppm H₂S, from about 5 vppm to about 7500 vppm H₂S, from about 5 vppm to about 5000 vppm H₂S, from about 5 vppm to about 2500 vppm H₂S, from about 5 vppm to about 1000 vppm H₂S, from about 5 vppm to about 500 vppm H₂S, from about 5 vppm to about 250 vppm H₂S, from about 5 vppm to about 100 vppm H₂S, from about 5 vppm to about 75 vppm H₂S, from about 5 vppm to about 50 vppm H₂S, from about 5 vppm to about 25 vppm H₂S, from about 5 vppm to about 10 vppm H₂S, from about 10 vppm to about 10000 vppm H₂S, from about 10 vppm to about 7500 vppm H₂S, from about 10 vppm to about 5000 vppm H₂S, from about 10 vppm to about 2500 vppm H₂S, from about 10 vppm to about 1000 vppm H₂S, from about 10 vppm to about 500 vppm H₂S, from about 10 vppm to about 250 vppm H₂S, from about 10 vppm to about 100 vppm H₂S, from about 10 vppm to about 75 vppm H₂S, from about 10 vppm to about 50 vppm H₂S, from about 10 vppm to about 25 vppm H₂S, from about 25 vppm to about 10000 vppm H₂S, from about 25 vppm to about 7500 vppm H₂S, from about 25 vppm to about 5000 vppm H₂S, from about 25 vppm to about 2500 vppm H₂S, from about 25 vppm to about 1000 vppm H₂S, from about 2 vppm to about 500 vppm H₂S, from about 25 vppm to about 250 vppm H₂S, from about 25 vppm to about 100 vppm H₂S, from about 25 vppm to about 75 vppm H₂S, from about 25 vppm to about 50 vppm H₂S, from about 50 vppm to about 10000 vppm H₂S, from about 50 vppm to about 7500 vppm H₂S, from about 50 vppm to about 5000 vppm H₂S, from about 50 vppm to about 2500 vppm H₂S, from about 50 vppm to about 1000 vppm H₂S, from about 50 vppm to about 500 vppm H₂S, from about 50 vppm to about 250 vppm H₂S, from about 50 vppm to about 100 vppm H₂S, from about 50 vppm to about 75 vppm H₂S, from about 75 vppm to about 10000 vppm H₂S, from about 100 vppm to about 10000 vppm H₂S, from about 100 vppm to about 7500 vppm H₂S, from about 100 vppm to about 5000 vppm H₂S, from about 100 vppm to about 2500 vppm H₂S, from about 100 vppm to about 1000 vppm H₂S, from about 100 vppm to about 500 vppm H₂S, from about 100 vppm to about 250 vppm H₂S, from about 500 vppm to about 10000 vppm H₂S, from about 500 vppm to about 7500 vppm H₂S, from about 500 vppm to about 5000 vppm H₂S, from about 500 vppm to about 2500 vppm H₂S, from about 500 vppm to about 1000 vppm H₂S, from about 1000 vppm to about 10000 vppm H₂S, from about 1000 vppm to about 5000 vppm H₂S, from about 1000 vppm to about 2500 vppm H₂S, from about 2500 vppm to about 10000 vppm H₂S, from about 2500 vppm to about 5000 vppm H₂S, or from about 5000 vppm to about 10000 vppm H₂S), and/or from about 5 vppm to about 25 vol % CO (e.g., from about 5 vppm to about 20 vol % CO, from about 5 vppm to about 10 vol % CO, from about 5 vppm to about 5 vol % CO, from about 5 vppm to about 3 vol % CO, from about 5 vppm to about 2 vol % CO, from about 5 vppm to about 1 vol % CO, from about 5 vppm to about 5000 vppm CO, from about 5 vppm to about 3000 vppm CO, from about 5 vppm to about 1000 vppm CO, from about 5 vppm to about 500 vppm CO, from about 5 vppm to about 300 vppm CO, from about 5 vppm to about 100 vppm CO, from about 5 vppm to about 50 vppm CO, from about 10 vppm to about 25 vol % CO, from about 10 vppm to about 20 vol % CO, from about 10 vppm to about 10 vol % CO, from about 10 vppm to about 5 vol % CO, from about 10 vppm to about 3 vol % CO, from about 10 vppm to about 2 vol % CO, from about 10 vppm to about 1 vol % CO, from about 10 vppm to about 5000 vppm CO, from about 10 vppm to about 3000 vppm CO, from about 10 vppm to about 1000 vppm CO, from about 10 vppm to about 500 vppm CO, from about 10 vppm to about 300 vppm CO, from about 10 vppm to about 100 vppm CO, from about 10 vppm to about 50 vppm CO, from about 50 vppm to about 25 vol % CO, from about 50 vppm to about 20 vol % CO, from about 50 vppm to about 10 vol % CO, from about 50 vppm to about 5 vol % CO, from about 50 vppm to about 3 vol % CO, from about 50 vppm to about 2 vol % CO, from about 50 vppm to about 1 vol % CO, from about 50 vppm to about 5000 vppm CO, from about 50 vppm to about 3000 vppm CO, from about 50 vppm to about 1000 vppm CO, from about 50 vppm to about 500 vppm CO, from about 50 vppm to about 300 vppm CO, from about 50 vppm to about 100 vppm CO, from about 100 vppm to about 25 vol % CO, from about 100 vppm to about 20 vol % CO, from about 100 vppm to about 10 vol % CO, from about 100 vppm to about 5 vol % CO, from about 100 vppm to about 3 vol % CO, from about 100 vppm to about 2 vol % CO, from about 100 vppm to about 1 vol % CO, from about 100 vppm to about 5000 vppm CO, from about 100 vppm to about 3000 vppm CO, from about 100 vppm to about 1000 vppm CO, from about 100 vppm to about 500 vppm CO, from about 500 vppm to about 25 vol % CO, from about 500 vppm to about 20 vol % CO, from about 500 vppm to about 10 vol % CO, from about 500 vppm to about 5 vol % CO, from about 500 vppm to about 3 vol % CO, from about 500 vppm to about 2 vol % CO, from about 500 vppm to about 1 vol % CO, from about 500 vppm to about 5000 vppm CO, from about 500 vppm to about 3000 vppm CO, from about 500 vppm to about 1000 vppm CO, from about 1000 vppm to about 25 vol % CO, from about 1000 vppm to about 20 vol % CO, from about 1000 vppm to about 10 vol % CO, from about 1000 vppm to about 5 vol % CO, from about 1000 vppm to about 3 vol % CO, from about 1000 vppm to about 2 vol % CO, from about 1000 vppm to about 1 vol % CO, from about 1000 vppm to about 5000 vppm CO, from about 1000 vppm to about 3000 vppm CO, from about 5000 vppm to about 25 vol % CO, from about 5000 vppm to about 20 vol % CO, from about 5000 vppm to about 10 vol % CO, from about 5000 vppm to about 5 vol % CO, from about 5000 vppm to about 3 vol % CO, from about 5000 vppm to about 2 vol % CO, from about 5000 vppm to about 1 vol % CO, from about 1 vol % to about 25 vol % CO, from about 1 vol % to about 20 vol % CO, from about 1 vol % to about 10 vol % CO, from about 1 vol % to about 5 vol % CO, or from about 1 vol % to about 3 vol % CO).

In many embodiments, the source(s) of mixed gas can (collectively and/or each) comprise at least one of the following: from about 1 vol % to about 25 vol % CO₂ and from about 0.5 vol % to about 20 vol % moisture; from about 10 vol % to about 45 vol % CO₂ and at least about 10 vol % C₁-C₃ hydrocarbons; from about 5 vppm to about 1000 vppm SOₓ; from about 5 vppm to about 1000 vppm NOₓ; from about 1 vol % to about 40 vol % H₂; from about 10 vppm to about 4000 vppm H₂S; and from about 50 vppm to about 5 vol % CO.

Alternately, in embodiments where one or more of the at least two solid monolithic sorbents are especially sensitive to the presence of one or more of SOₓ, NOₓ, H₂S, and CO (e.g., where such component(s) substantially shorten(s) sorbent useful life, substantially reduce(s) sorbent activity, substantially reduce(s) sorbent selectivity for the target gas component(s), substantially detrimentally affect(s) sorbent structural and/or chemical stability, or the like, or a combination thereof), the individual content of the sensitive compound(s) in the collective mixed gas source can be, or can be pre-treated to be, about 50 vppm or less, e.g., about 40 vppm or less, about 30 vppm or less, about 20 vppm or less, about 10 vppm or less, about 7 vppm or less, about 5 vppm or less, about 3 vppm or less, about 2 vppm or less, about 1 vppm or less, about 750 vppb or less, about 500 vppb or less, about 250 vppb or less, about 100 vppb or less, about 75 vppb or less, about 50 vppb or less, or about 25 vppb or less. In such alternate component-sensitive embodiments, though there may not necessarily be a lower limit on each sensitive component content, it can be practically very difficult to achieve (and/or to experimentally measure) contents below about 10 vppb. A non-limiting example of a SOₓ-sensitive sorbent material can include solid/grafted amine sorbents, or sorbents having a functionality exhibiting Lewis basicity, such as containing a nitrogen atom with a lone pair of electrons.

Instead of characterizing the source(s) of mixed gas by the relative contents of their (respective/collective) components, they can additionally or alternately be characterized by their origin. For example, the source(s) of mixed gas can collectively and/or each include (or be comprised of) a petroleum refinery flue gas stream, product and/or waste from a coal-burning power plant, a water gas shift process product stream, a hydrocarbon conversion catalyst regeneration gas, a hydrocarbon combustion gas product stream, a virgin or partially treated natural gas stream, or a combination thereof.

The nature of the at least two solid monolithic sorbents can vary, depending upon the specific desired component(s) to be controlled, separated, and/or purified. In many embodiments, one or more of the at least two solid monolithic sorbents can comprise or be formed from: an alkalized alumina; an alkalized titania; activated carbon; 13X or 5A molecular sieve; a mesoporous molecular sieve material such as MCM-48; a zeolite having framework structure type AEI, AFT, AFX, ATN, AWW, CHA, DDR, EPI, ESV, FAU, KFI, LEV, LTA, PHI, RHO, SAV, or a combination or intergrowth thereof; a cationic zeolite material; a metal oxide whose metal(s) include(s) an alkali metal, an alkaline earth metal, a transition metal, or a combination thereof; a zeolite imidazolate framework (ZIF) material; a metal organic framework (MOF) material; or a combination thereof. In a preferred embodiment where carbon dioxide is to be separated from a mixed gas containing at least carbon dioxide, some C₁-C₃ hydrocarbons, and some moisture, the at least two solid monolithic sorbents can advantageously be formed from an alkalized alumina. Additionally or alternately, any of the solid monolithic sorbents used in methods according to the invention can be functionalized (e.g., on one or more surfaces exposed to the carbon oxide-containing gas flow) with sorbent functional groups, including chemisorptive functional groups such as primary and/or secondary amines.

The sorption-desorption process can typically include at least the following steps: first and second CO₂ sorption; sorbent heating; first and second CO₂ desorption; and sorbent cooling. Though these steps are detailed in an order from first to last, it should be understood that this is only for convenience of explanation and is not meant to unduly limit the present invention; for instance, as described further herein, the stated order of these steps from first to last is not necessarily the order in which they would occur in the methods according to the invention. Furthermore, since at least two solid monolithic sorbents are being used, it can be convenient to refer herein to steps as occurring simultaneously in both sorbents, but it should be understood from the further disclosure that, though the steps may be similar, the process occurring with respect to each sorbent may be described as beginning/ending in a different place or in the same place within the process as with respect to one or more other sorbents, without meaning to imply that they are necessarily occurring at different times or simultaneously, respectively.

In the first CO₂ sorption step, the mixed gas source(s), which contain(s) CO₂ at a first temperature, can be exposed to at least a portion of the solid monolithic sorbents, which are at a second temperature and under further conditions sufficient for the solid monolithic sorbents to selectively sorb the CO₂, which second temperature can be higher (particularly in TSA-type processes, e.g., at least about 10° C. higher, at least about 15° C. higher, at least about 20° C. higher, at least about 25° C. higher, at least about 30° C. higher, at least about 35° C. higher, at least about 40° C. higher, at least about 45° C. higher, or at least about 50° C. higher; additionally or alternately in such TSA-type processes, no more than about 100° C. higher, e.g., no more than about 90° C. higher, no more than about 85° C. higher, no more than about 80° C. higher, no more than about 75° C. higher, no more than about 70° C. higher, no more than about 65° C. higher, no more than about 60° C. higher, no more than about 55° C. higher, no more than about 50° C. higher, no more than about 45° C. higher, no more than about 40° C. higher, no more than about 35° C. higher, no more than about 30° C. higher, no more than about 25° C. higher, or no more than about 20° C. higher) than the first temperature. Alternately, in certain embodiments, the second temperature can be cooler than the first temperature. As a result, at least partially, selectively CO₂-sorbed solid monolithic sorbents can be formed, along with an at least partially, selectively CO₂-depleted product stream. As a consequence of the formation (e.g., due to the exothermic nature of the sorption process), the aforementioned portion of the solid monolithic sorbents can be simultaneously heated to a third temperature higher than the second temperature.

In an optional embodiment, the aforementioned portion of the selectively-CO₂-sorbed solid monolithic sorbents can be further heated to a fourth temperature above the third temperature in the second CO₂ sorption step, in order to facilitate more efficient desorption. In such optional embodiments, the fourth temperature can be at least about 3° C. higher, e.g., at least about 5° C. higher, at least about 10° C. higher, at least about 15° C. higher, at least about 20° C. higher, at least about 25° C. higher, at least about 30° C. higher, at least about 35° C. higher, at least about 40° C. higher, at least about 45° C. higher, or at least about 50° C. higher, than the third temperature. Additionally or alternately, in such optional embodiments, the third temperature can be no more than about 80° C. higher, e.g., no more than about 75° C. higher, no more than about 70° C. higher, no more than about 65° C. higher, no more than about 60° C. higher, no more than about 55° C. higher, no more than about 50° C. higher, no more than about 45° C. higher, no more than about 40° C. higher, no more than about 35° C. higher, no more than about 30° C. higher, no more than about 25° C. higher, no more than about 20° C. higher, no more than about 15° C. higher, no more than about 10° C. higher, or no more than about 5° C. higher, than the third temperature. However, in other embodiments, this optional further heating step is not conducted.

In the first CO₂ desorption step, the CO₂-sorbed and heated solid monolithic sorbents can be exposed to a countercurrent at least partially stripped product stream containing desorbed CO₂ and moisture. As a result, at least partially CO₂-desorbed and heated monolithic sorbents can be formed, along with a further stripped product stream containing additional desorbed CO₂ and having a lower moisture content than that of the at least partially stripped product stream. As a consequence of this desorption step (e.g., though the desorption process can typically be endothermic in nature, that endotherm can typically be overwhelmed, at least in TSA-type processes, by the greater temperature differential between the desorption stream and the CO₂-sorbed solid monolithic sorbents), the at least partially CO₂-desorbed and heated monolithic sorbents can be simultaneously further heated to a fifth temperature above the third (or optional fourth) temperature and can additionally thus contain moisture. In such embodiments, the fifth temperature can be at least about 3° C. higher, e.g., at least about 5° C. higher, at least about 10° C. higher, at least about 15° C. higher, at least about 20° C. higher, at least about 25° C. higher, at least about 30° C. higher, at least about 35° C. higher, at least about 40° C. higher, at least about 45° C. higher, at least about 50° C. higher, at least about 55° C. higher, at least about 60° C. higher, at least about 65° C. higher, at least about 70° C. higher, at least about 75° C. higher, at least about 80° C. higher, at least about 85° C. higher, or at least about 90° C. higher, than the third (or optional fourth) temperature. Additionally or alternately, the fifth temperature can be no more than about 100° C. higher, e.g., no more than about 90° C. higher, no more than about 85° C. higher, no more than about 80° C. higher, no more than about 75° C. higher, no more than about 70° C. higher, no more than about 65° C. higher, no more than about 60° C. higher, no more than about 55° C. higher, no more than about 50° C. higher, no more than about 45° C. higher, no more than about 40° C. higher, no more than about 35° C. higher, no more than about 30° C. higher, no more than about 25° C. higher, no more than about 20° C. higher, no more than about 15° C. higher, no more than about 10° C. higher, or no more than about 5° C. higher, than the third (or optionally fourth) temperature.

In the second CO₂ desorption step, the at least partially CO₂-desorbed and heated solid monolithic sorbents can be exposed to a countercurrent CO₂ stripping stream, typically containing moisture and a relatively low CO₂ content (preferably not more than about 2 vol % CO₂, e.g., not more than about 1 vol % CO₂, not more than about 5000 vppm CO₂, not more than about 3000 vppm CO₂, not more than about 2000 vppm CO₂, not more than about 1000 vppm CO₂, not more than about 750 vppm CO₂, not more than about 500 vppm CO₂, not more than about 300 vppm CO₂, not more than about 200 vppm CO₂, not more than about 100 vppm CO₂, not more than about 75 vppm CO₂, not more than about 50 vppm CO₂, not more than about 30 vppm CO₂, not more than about 20 vppm CO₂, or not more than about 10 vppm CO₂) to facilitate further desorption of CO₂. As a result, further CO₂-desorbed and heated monolithic sorbents can be formed, as well as an at least partially stripped product stream containing desorbed CO₂ and moisture, which can preferably be used in the first CO₂ gas desorption step. As a consequence of this desorption step (e.g., though the desorption process can typically be endothermic in nature, that endotherm can typically be overwhelmed, at least in TSA-type processes, by the greater temperature differential between the desorption stream and the CO₂-sorbed solid monolithic sorbents), the further CO₂-desorbed and heated monolithic sorbents can be further heated to a sixth temperature, which can typically be at or among the highest temperature that the sorbents achieve in the system, and can thus be higher than the fifth temperature, as well as containing additional moisture. In certain embodiments, the sixth temperature can be at least about 3° C. higher, e.g., at least about 5° C. higher, at least about 10° C. higher, at least about 15° C. higher, at least about 20° C. higher, at least about 25° C. higher, at least about 30° C. higher, at least about 35° C. higher, at least about 40° C. higher, at least about 45° C. higher, or at least about 50° C. higher, than the fifth temperature. Additionally or alternately, in such embodiments, the sixth temperature can be no more than about 80° C. higher, e.g., no more than about 75° C. higher, no more than about 70° C. higher, no more than about 65° C. higher, no more than about 60° C. higher, no more than about 55° C. higher, no more than about 50° C. higher, no more than about 45° C. higher, no more than about 40° C. higher, no more than about 35° C. higher, no more than about 30° C. higher, no more than about 25° C. higher, no more than about 20° C. higher, no more than about 15° C. higher, no more than about 10° C. higher, or no more than about 5° C. higher, than the fifth temperature.

In the sorbent cooling step, the further CO₂-desorbed and heated monolithic sorbents can be exposed to a cooling stream at a seventh temperature, which can typically be at or among the lowest temperature that the sorbents achieve in the system, and can thus be lower than the second temperature. As a result of the cooling step, the solid monolithic sorbents can be cooled to an eighth temperature (typically not quite equal to and a bit higher than the seventh temperature, but usually still lower than the second temperature). In such embodiments, the eighth temperature can be at least about 3° C. lower, e.g., at least about 5° C. lower, at least about 7° C. lower, at least about 10° C. lower, at least about 15° C. lower, at least about 20° C. lower, at least about 25° C. lower, at least about 30° C. lower, at least about 35° C. lower, at least about 40° C. lower, at least about 45° C. lower, or at least about 50° C. lower, than the second temperature. Additionally or alternately in such embodiments, the eighth temperature can be no more than about 75° C. lower, e.g., no more than about 70° C. lower, no more than about 65° C. lower, no more than about 60° C. lower, no more than about 55° C. lower, no more than about 50° C. lower, no more than about 45° C. lower, no more than about 40° C. lower, no more than about 35° C. lower, no more than about 30° C. lower, no more than about 25° C. lower, no more than about 20° C. lower, no more than about 15° C. lower, no more than about 10° C. lower, no more than about 7° C. lower, or no more than about 5° C. lower, than the second temperature.

In an optional embodiment, simultaneously with the sorbent cooling step, subsequent to the sorbent cooling step, and/or prior to the second CO₂ sorption step, the monolithic sorbents can be exposed to a further drying stream to thus form cooled and dried monolithic sorbents having sorbed moisture and to thus also form a drying throughput stream. In some such optional embodiments, at least a portion of the drying throughput stream can optionally be recycled to the source(s) of mixed gas used in the first CO₂ sorption step. However, in some embodiments, there is no optional drying step between the sorbent cooling step and the second CO₂ sorption step.

In the second CO₂ sorption step, the at least partially, selectively CO₂-depleted product stream from the first CO₂ sorption step can be exposed to the cooled (and optionally dried) solid monolithic sorbents under conditions sufficient for the cooled (and optionally dried) monolithic sorbents to selectively sorb additional CO₂ gas from the at least partially CO₂-depleted product stream. As a result, the at least partially CO₂-sorbed solid monolithic sorbents can be formed, along with a further CO₂-depleted product stream. As a consequence of this sorption step (e.g., due to the exothermic nature of the sorption process), the solid monolithic sorbents can be simultaneously heated to the second temperature. Obviously, as the methods according to the present invention are envisioned to be continuous (or at least semi-continuous), this sorption-desorption cycle can advantageously be repeated.

In an optional embodiment, moisture from the at least partially stripped product stream and/or from the further stripped product stream can be condensed as water, thus forming one or more condensed product streams and thereby increasing CO₂ concentration/purity in the uncondensed product stream (the at least partially stripped product stream without the condensed water). The at least partially stripped product stream, the further stripped product stream, the uncondensed product stream, and/or the condensed product stream(s) can optionally be further processed, if desired, and/or can optionally be used, in whole or in part, as an integration with one or more chemical, refinery, CO₂ sequestration, gas production, and/or other industrial/commercial process.

In one, some, or all of the CO₂ sorption steps (e.g., in the first and/or second CO₂ sorption steps), the solid monolithic sorbents (e.g., on one, more than one, or each rotary wheel) can optionally but advantageously have a CO₂/N₂ selectivity at the operating conditions in the sorption steps of at least 2, e.g., at least 3, at least 4, at least 5, at least 7, at least 10, at least 15, at least 20, at least 25, at least 30, at least 50, at least 75, at least 100, at least 200, at least 300, at least 400, at least 500, at least 750, or at least 1000. Additionally or alternately, the solid monolithic sorbents can optionally but advantageously have a CO₂/N₂ selectivity at the operating conditions in the sorption steps of up to 10000, e.g., up to 7500, up to 5000, up to 3000, up to 2500, up to 2000, up to 1500, up to 1000, up to 750, up to 500, up to 300, up to 250, up to 200, up to 150, up to 100, up to 75, up to 50, up to 30, up to 25, up to 20, up to 15, or up to 10.

In one, some, or all of the CO₂ sorption steps (e.g., in the first and/or second CO₂ sorption steps), the solid monolithic sorbents (e.g., on one, more than one, or each rotary wheel) can optionally have a CO₂/CH₄ selectivity at the operating conditions in the sorption steps of at least 2, e.g., at least 3, at least 4, at least 5, at least 7, at least 10, at least 15, at least 20, at least 25, at least 30, at least 50, at least 75, at least 100, at least 200, at least 300, at least 400, at least 500, at least 750, or at least 1000. Additionally or alternately, the solid monolithic sorbents can optionally but advantageously have a CO₂/CH₄ selectivity at the operating conditions in the sorption steps of up to 10000, e.g., up to 7500, up to 5000, up to 3000, up to 2500, up to 2000, up to 1500, up to 1000, up to 750, up to 500, up to 300, up to 250, up to 200, up to 150, up to 100, up to 75, up to 50, up to 30, up to 25, up to 20, up to 15, or up to 10.

In certain embodiments, the cyclic sorption-desorption process can have an average total cycle time from about 30 seconds to about 720 minutes, e.g., from about 30 seconds to about 600 minutes, from about 30 seconds to about 480 minutes, from about 30 seconds to about 360 minutes, from about 30 seconds to about 240 minutes, from about 30 seconds to about 180 minutes, from about 30 seconds to about 120 minutes, from about 30 seconds to about 90 minutes, from about 30 seconds to about 60 minutes, from about 30 seconds to about 45 minutes, from about 30 seconds to about 30 minutes, from about 30 seconds to about 20 minutes, from about 30 seconds to about 15 minutes, from about 30 seconds to about 10 minutes, from about 30 seconds to about 5 minutes, from about 1 minute to about 720 minutes, from about 1 minute to about 600 minutes, from about 1 minute to about 480 minutes, from about 1 minute to about 360 minutes, from about 1 minute to about 240 minutes, from about 1 minute to about 180 minutes, from about 1 minute to about 120 minutes, from about 1 minute to about 90 minutes, from about 1 minute to about 60 minutes, from about 1 minute to about 45 minutes, from about 1 minute to about 30 minutes, from about 1 minute to about 20 minutes, from about 1 minute to about 15 minutes, from about 1 minute to about 10 minutes, from about 1 minute to about 5 minutes, from about 3 minutes to about 720 minutes, from about 3 minutes to about 600 minutes, from about 3 minutes to about 480 minutes, from about 3 minutes to about 360 minutes, from about 3 minutes to about 240 minutes, from about 3 minutes to about 180 minutes, from about 3 minutes to about 120 minutes, from about 3 minutes to about 90 minutes, from about 3 minutes to about 60 minutes, from about 3 minutes to about 45 minutes, from about 3 minutes to about 30 minutes, from about 3 minutes to about 20 minutes, from about 3 minutes to about 15 minutes, from about 3 minutes to about 10 minutes, from about 5 minutes to about 720 minutes, from about 5 minutes to about 600 minutes, from about 5 minutes to about 480 minutes, from about 5 minutes to about 360 minutes, from about 5 minutes to about 240 minutes, from about 5 minutes to about 180 minutes, from about 5 minutes to about 120 minutes, from about 5 minutes to about 90 minutes, from about 5 minutes to about 60 minutes, from about 5 minutes to about 45 minutes, from about 5 minutes to about 30 minutes, from about 5 minutes to about 20 minutes, from about 5 minutes to about 15 minutes, from about 5 minutes to about 10 minutes, from about 10 minutes to about 720 minutes, from about 10 minutes to about 600 minutes, from about 10 minutes to about 480 minutes, from about 10 minutes to about 360 minutes, from about 10 minutes to about 240 minutes, from about 10 minutes to about 180 minutes, from about 10 minutes to about 120 minutes, from about 10 minutes to about 90 minutes, from about 10 minutes to about 60 minutes, from about 10 minutes to about 45 minutes, from about 10 minutes to about 30 minutes, from about 10 minutes to about 20 minutes, from about 15 minutes to about 720 minutes, from about 15 minutes to about 600 minutes, from about 15 minutes to about 480 minutes, from about 15 minutes to about 360 minutes, from about 15 minutes to about 240 minutes, from about 15 minutes to about 180 minutes, from about 15 minutes to about 120 minutes, from about 15 minutes to about 90 minutes, from about 15 minutes to about 60 minutes, from about 15 minutes to about 45 minutes, from about 15 minutes to about 30 minutes, from about 20 minutes to about 720 minutes, from about 20 minutes to about 600 minutes, from about 20 minutes to about 480 minutes, from about 20 minutes to about 360 minutes, from about 20 minutes to about 240 minutes, from about 20 minutes to about 180 minutes, from about 20 minutes to about 120 minutes, from about 20 minutes to about 90 minutes, from about 20 minutes to about 60 minutes, from about 20 minutes to about 45 minutes, from about 20 minutes to about 30 minutes, from about 30 minutes to about 720 minutes, from about 30 minutes to about 600 minutes, from about 30 minutes to about 480 minutes, from about 30 minutes to about 360 minutes, from about 30 minutes to about 240 minutes, from about 30 minutes to about 180 minutes, from about 30 minutes to about 120 minutes, from about 30 minutes to about 90 minutes, from about 30 minutes to about 60 minutes, or from about 30 minutes to about 45 minutes.

In most embodiments, the conditions sufficient for one, some, or all of the (e.g., for the first and/or second) CO₂ desorption steps can include a pressure swing/reduction, a temperature swing/increase, or both. As such, the cyclic sorption-desorption methods according to the invention can involve PSA, rapid cycle PSA (RCPSA), TSA, rapid cycle TSA (RCTSA), a combination of pressure and temperature swings (PTSA), a partial pressure swing (PPSA), or the like, or some combination or integration thereof. In embodiments where rapid cycles are desired/utilized, the average total cycle time can be no more than about 1 minute, e.g., no more than about 45 seconds, no more than about 30 seconds, no more than about 20 seconds, no more than about 15 seconds, no more than about 10 seconds, or no more than about 5 seconds (and, though no lower limit is specified, it can be impractical in some embodiments for solid monolithic sorbents to undergo an average total cycle time less than about 1 second).

In many embodiments, the total pressure conditions in one, some, or all of the (e.g., in the first and/or second) CO₂ sorption steps, in the sorbent heating step, in one, some, or all of the (e.g., in the first and/or second) CO₂ desorption steps, in the sorbent cooling step, and/or in any one or more of the optional steps (when present) of the sorption-desorption process can collectively range from about 0.01 psia (about 0.07 kPaa) to about 300 psia (about 2.0 MPaa), e.g., from about 0.01 psia (about 0.07 kPaa) to about 200 psia (about 1.4 MPaa), from about 0.01 psia (about 0.07 kPaa) to about 150 psia (about 1.0 MPaa), from about 0.01 psia (about 0.07 kPaa) to about 100 psia (about 690 kPaa), from about 0.01 psia (about 0.07 kPaa) to about 75 psia (about 520 kPaa), from about 0.01 psia (about 0.07 kPaa) to about 60 psia (about 410 kPaa), from about 0.01 psia (about 0.07 kPaa) to about 50 psia (about 340 kPaa), from about 0.01 psia (about 0.07 kPaa) to about 40 psia (about 280 kPaa), from about 0.01 psia (about 0.07 kPaa) to about 30 psia (about 210 kPaa), from about 0.01 psia (about 0.07 kPaa) to about 25 psia (about 170 kPaa), from about 0.01 psia (about 0.07 kPaa) to about 20 psia (about 140 kPaa), from about 0.01 psia (about 0.07 kPaa) to about 15 psia (about 100 kPaa), from about 0.1 psia (about 0.7 kPaa) to about 300 psia (about 2.0 MPaa), from about 0.1 psia (about 0.7 kPaa) to about 200 psia (about 1.4 MPaa), from about 0.1 psia (about 0.7 kPaa) to about 150 psia (about 1.0 MPaa), from about 0.1 psia (about 0.7 kPaa) to about 100 psia (about 690 kPaa), from about 0.1 psia (about 0.7 kPaa) to about 75 psia (about 520 kPaa), from about 0.1 psia (about 0.7 kPaa) to about 60 psia (about 410 kPaa), from about 0.1 psia (about 0.7 kPaa) to about 50 psia (about 340 kPaa), from about 0.1 psia (about 0.7 kPaa) to about 40 psia (about 280 kPaa), from about 0.1 psia (about 0.7 kPaa) to about 30 psia (about 210 kPaa), from about 0.1 psia (about 0.7 kPaa) to about 25 psia (about 170 kPaa), from about 0.1 psia (about 0.7 kPaa) to about 20 psia (about 140 kPaa), from about 0.1 psia (about 0.7 kPaa) to about 15 psia (about 100 kPaa), from about 1 psia (about 7 kPaa) to about 300 psia (about 2.0 MPaa), from about 1 psia (about 7 kPaa) to about 200 psia (about 1.4 MPaa), from about 1 psia (about 7 kPaa) to about 150 psia (about 1.0 MPaa), from about 1 psia (about 7 kPaa) to about 100 psia (about 690 kPaa), from about 1 psia (about 7 kPaa) to about 75 psia (about 520 kPaa), from about 1 psia (about 7 kPaa) to about 60 psia (about 410 kPaa), from about 1 psia (about 7 kPaa) to about 50 psia (about 340 kPaa), from about 1 psia (about 7 kPaa) to about 40 psia (about 280 kPaa), from about 1 psia (about 7 kPaa) to about 30 psia (about 210 kPaa), from about 1 psia (about 7 kPaa) to about 25 psia (about 170 kPaa), from about 1 psia (about 7 kPaa) to about 20 psia (about 140 kPaa), from 1 psia (about 7 kPaa) to about 15 psia (about 100 kPaa), from about 10 psia (about 70 kPaa) to about 300 psia (about 2.0 MPaa), from about 10 psia (about 70 kPaa) to about 200 psia (about 1.4 MPaa), from about 10 psia (about 70 kPaa) to about 150 psia (about 1.0 MPaa), from about 10 psia (about 70 kPaa) to about 100 psia (about 690 kPaa), from about 10 psia (about 70 kPaa) to about 75 psia (about 520 kPaa), from about 10 psia (about 70 kPaa) to about 60 psia (about 410 kPaa), from about 10 psia (about 70 kPaa) to about 50 psia (about 340 kPaa), from about 10 psia (about 70 kPaa) to about 40 psia (about 280 kPaa), from about 10 psia (about 70 kPaa) to about 30 psia (about 210 kPaa), from about 10 psia (about 70 kPaa) to about 25 psia (about 170 kPaa), from about 10 psia (about 70 kPaa) to about 20 psia (about 140 kPaa), from 10 psia (about 70 kPaa) to about 15 psia (about 100 kPaa), from about 15 psia (about 100 kPaa) to about 300 psia (about 2.0 MPaa), from about 15 psia (about 100 kPaa) to about 200 psia (about 1.4 MPaa), from about 15 psia (about 100 kPaa) to about 150 psia (about 1.0 MPaa), from about 15 psia (about 100 kPaa) to about 100 psia (about 690 kPaa), from about 15 psia (about 100 kPaa) to about 75 psia (about 520 kPaa), from about 15 psia (about 100 kPaa) to about 60 psia (about 410 kPaa), from about 15 psia (about 100 kPaa) to about 50 psia (about 340 kPaa), from about 15 psia (about 100 kPaa) to about 40 psia (about 280 kPaa), from about 15 psia (about 100 kPaa) to about 30 psia (about 210 kPaa), from about 15 psia (about 100 kPaa) to about 25 psia (about 170 kPaa), or from about 15 psia (about 100 kPaa) to about 20 psia (about 140 kPaa).

In certain embodiments, the temperature conditions for all the input streams, output streams, and solid monolithic sorbents in one, some, or all of the (e.g., in the first and/or second) CO₂ sorption steps, in the sorbent heating step, in one, some, or all of the (e.g., in the first and/or second) CO₂ desorption steps, in the sorbent cooling step, and/or in any one or more of the optional steps (when present) of the sorption-desorption process can collectively range from about −40° C. to about 250° C., e.g., from about −25° C. to about 250° C., from about −10° C. to about 250° C., from about 0° C. to about 250° C., from about 5° C. to about 250° C., from about 10° C. to about 250° C., from about 15° C. to about 250° C., from about 20° C. to about 250° C., from about 25° C. to about 250° C., from about 30° C. to about 250° C., from about 35° C. to about 250° C., from about 40° C. to about 250° C., from about 45° C. to about 250° C., from about 50° C. to about 250° C., from about 60° C. to about 250° C., from about 70° C. to about 250° C., from about 80° C. to about 250° C., from about 90° C. to about 250° C., from about −40° C. to about 225° C., from about −25° C. to about 225° C., from about −10° C. to about 225° C., from about 0° C. to about 225° C., from about 5° C. to about 225° C., from about 10° C. to about 225° C., from about 15° C. to about 225° C., from about 20° C. to about 225° C., from about 25° C. to about 225° C., from about 30° C. to about 225° C., from about 35° C. to about 225° C., from about 40° C. to about 225° C., from about 45° C. to about 225° C., from about 50° C. to about 225° C., from about 60° C. to about 225° C., from about 70° C. to about 225° C., from about 80° C. to about 225° C., from about 90° C. to about 225° C., from about −40° C. to about 205° C., from about −25° C. to about 205° C., from about −10° C. to about 205° C., from about 0° C. to about 205° C., from about 5° C. to about 205° C., from about 10° C. to about 205° C., from about 15° C. to about 205° C., from about 20° C. to about 205° C., from about 25° C. to about 205° C., from about 30° C. to about 205° C., from about 35° C. to about 205° C., from about 40° C. to about 205° C., from about 45° C. to about 205° C., from about 50° C. to about 205° C., from about 60° C. to about 205° C., from about 70° C. to about 205° C., from about 80° C. to about 205° C., from about 90° C. to about 205° C., from about −40° C. to about 190° C., from about −25° C. to about 190° C., from about −10° C. to about 190° C., from about 0° C. to about 190° C., from about 5° C. to about 190° C., from about 10° C. to about 190° C., from about 15° C. to about 190° C., from about 20° C. to about 190° C., from about 25° C. to about 190° C., from about 30° C. to about 190° C., from about 35° C. to about 190° C., from about 40° C. to about 190° C., from about 45° C. to about 190° C., from about 50° C. to about 190° C., from about 60° C. to about 190° C., from about 70° C. to about 190° C., from about 80° C. to about 190° C., from about 90° C. to about 190° C., from about −40° C. to about 175° C., from about −25° C. to about 175° C., from about −10° C. to about 175° C., from about 0° C. to about 175° C., from about 5° C. to about 175° C., from about 10° C. to about 175° C., from about 15° C. to about 175° C., from about 20° C. to about 175° C., from about 25° C. to about 175° C., from about 30° C. to about 175° C., from about 35° C. to about 175° C., from about 40° C. to about 175° C., from about 45° C. to about 175° C., from about 50° C. to about 175° C., from about 60° C. to about 175° C., from about 70° C. to about 175° C., from about 80° C. to about 175° C., from about 90° C. to about 175° C., from about −40° C. to about 160° C., from about −25° C. to about 160° C., from about −10° C. to about 160° C., from about 0° C. to about 160° C., from about 5° C. to about 160° C., from about 10° C. to about 160° C., from about 15° C. to about 160° C., from about 20° C. to about 160° C., from about 25° C. to about 160° C., from about 30° C. to about 160° C., from about 35° C. to about 160° C., from about 40° C. to about 160° C., from about 45° C. to about 160° C., from about 50° C. to about 160° C., from about 60° C. to about 160° C., from about 70° C. to about 160° C., from about 80° C. to about 160° C., from about 90° C. to about 160° C., from about −40° C. to about 145° C., from about −25° C. to about 145° C., from about −10° C. to about 145° C., from about 0° C. to about 145° C., from about 5° C. to about 145° C., from about 10° C. to about 145° C., from about 15° C. to about 145° C., from about 20° C. to about 145° C., from about 25° C. to about 145° C., from about 30° C. to about 145° C., from about 35° C. to about 145° C., from about 40° C. to about 145° C., from about 45° C. to about 145° C., from about 50° C. to about 145° C., from about 60° C. to about 145° C., from about 70° C. to about 145° C., from about 80° C. to about 145° C., or from about 90° C. to about 145° C.

Aside from the stripping streams that function to desorb at least a portion of the CO₂ in the desorption steps, additional regeneration of adsorbent materials may be carried out periodically, as necessary to achieve appropriate sorption and desorption performance under the methods according to the invention. The periodic additional regeneration may be regular (e.g., every cycle, every certain number of cycles, every certain number of days or months, or the like) and/or irregular (e.g., when one or more aspects of the methods according to the invention become difficult or impractical and/or upon failure of one or more aspects of the methods according to the invention such as lack of fluid communication, operation outside of a desired specification, or the like, or a combination thereof), inter alia. Additional (non-stripping) regeneration of sorbent materials can include, but is not necessarily limited to, induction heating and/or microwave irradiation. In the case of an sorbent monolith configured to rotate on a central axis, the mechanism of microwave irradiation can, in some embodiments, result in an internal heating emanating from one or more appropriately placed microwave antennae, e.g., axially and radially outward therefrom. Additionally or alternately in the case of a sorbent monolith, induction heating can, in many embodiments, result in an external heating emanating inward from the induction source, e.g., such that the skin/surface of the monolith is rapidly heated, with the heat being transferred axially and radially inward through the remainder of the monolith.

In a preferred embodiment, the cyclic sorption-desorption process can comprise exactly two solid monolithic sorbents, a first and a second, and thus two sets of streams for each step, also a first and a second. In this preferred embodiment, the first CO₂ sorption step can include exposing the first mixed gas source to the first solid monolithic sorbent to form the first at least partially CO₂-sorbed solid monolithic sorbent and the first at least partially CO₂-depleted product stream, and exposing the second mixed gas source to the second solid monolithic sorbent to form the second at least partially CO₂-sorbed solid monolithic sorbent and the second at least partially CO₂-depleted product stream. Additionally in this preferred embodiment, the first at least partially CO₂-depleted product stream from the first CO₂ sorption step can then be exposed to the second cooled and optionally dried monolithic sorbent in the second CO₂ sorption step, thus forming the second further CO₂-depleted product stream, and the second at least partially CO₂-depleted product stream from the first CO₂ sorption step can then be exposed to the first cooled and optionally dried monolithic sorbent in the second CO₂ sorption step, thus forming the first further CO₂-depleted product stream. Further in this preferred embodiment, the second CO₂ desorption step can include exposing the first CO₂ stripping stream to the first at least partially CO₂-desorbed and heated solid monolithic sorbent to form the first further CO₂-desorbed and heated solid monolithic sorbent and the first at least partially stripped product stream, and exposing the second CO₂ stripping stream to the second at least partially CO₂-desorbed and heated solid monolithic sorbent to form the second further CO₂-desorbed and heated solid monolithic sorbent and the second at least partially stripped product stream. Still further in this preferred embodiment, the first at least partially stripped product stream from the second CO₂ desorption step can then be exposed to the second CO₂-sorbed and heated solid monolithic sorbent in the first CO₂ desorption step, thus forming the second further stripped product stream, and the second at least partially stripped product stream from the second CO₂ desorption step can then be exposed to the first CO₂-sorbed and heated solid monolithic sorbent in the first CO₂ desorption step, thus forming the first further stripped product stream.

In another preferred embodiment, the cyclic sorption-desorption process can comprise the at least two solid monolithic sorbents each radially rotating about a rotational axis, such that each solid monolithic sorbent is independent of the other(s). In this other preferred embodiment, the first CO₂ sorption step can include exposing each mixed gas source to its corresponding solid monolithic sorbent to form its corresponding at least partially CO₂-sorbed solid monolithic sorbent and its corresponding at least partially CO₂-depleted product stream. Additionally in this other preferred embodiment, each at least partially CO₂-depleted product stream from the first CO₂ sorption step can then be exposed to its corresponding cooled and optionally dried monolithic sorbent in the second CO₂ sorption step, thus forming its corresponding further CO₂-depleted product stream. Further in this other preferred embodiment, the second CO₂ desorption step can include exposing each CO₂ stripping stream to its corresponding at least partially CO₂-desorbed and heated solid monolithic sorbent to form its corresponding further CO₂-desorbed and heated solid monolithic sorbent and its corresponding at least partially stripped product stream. Still further in this other preferred embodiment, each at least partially stripped product stream from the second CO₂ desorption step can then be exposed to its corresponding CO₂-sorbed and heated solid monolithic sorbent in the first CO₂ desorption step, thus forming its corresponding further stripped product stream.

Alternately, in one, some, or all of the CO₂ sorption steps, the advantageous methods according to the invention can make it possible to use solid monolithic sorbents (collectively and/or each) having a CO₂ to specific contaminant (e.g., CO₂/N₂, CO₂/CH₄, or the like) selectivity of 3 or less, e.g., 2.5 or less, 2 or less, from 1 to 3, from 1.2 to 3, from 1.4 to 3, from 1.6 to 3, from 1.8 to 3, from 2 to 3, from 1 to 2.5, from 1.2 to 2.5, from 1.6 to 2.5, from 1.8 to 2.5, from 2 to 2.5, from 1 to 2, from 1.2 to 2, from 1.4 to 2, or from 1.6 to 2. In such embodiments, the use of relatively unselective sorbent material(s) can be used to attain efficiencies, yields, purities, and/or other improvements flowing from the methods according to the invention that would have required relatively selective (or at least significantly higher selectivity) sorbent material(s) to be used in otherwise identical single-monolith processes, otherwise identical multiple-monolith processes using co-current and/or co-rotating monoliths, or the like.

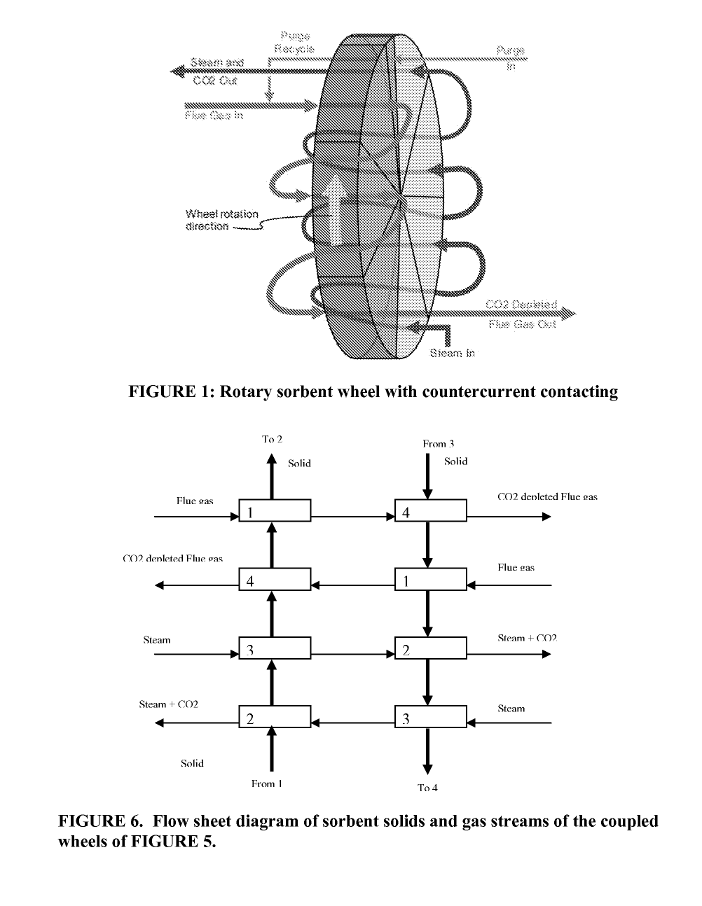

FIG. 1 illustrates the general concept of countercurrent contacting of the flue gas with the sorbent in a single wheel configuration—this countercurrent concept can be expanded to multiple wheel systems. FIG. 1 shows a wheel sector for adsorption (green) divided into three subsections or stages, with a desorption sector (blue) divided into four subsections or stages. It should be noted that the three and four stages are for illustration only and a fewer or larger number of stages may be used within each sector.

The configuration of FIG. 1 can allow for effectively countercurrent contacting for sorption and desorption. The incoming flue gas can contact a sorbent stage that had already contacted the gas feed source (e.g., flue gas) previously. Thus, for the overall sorption sector, the sorbent can see an increasing concentration of CO₂ as it rotates from the desorption sector to the gas feed. This contacting of the sorbent with an increasing CO₂ concentration in the gas can lead to an increased CO₂ concentration on the sorbent. This in turn can lead to improved CO₂ separation (higher purity and recovery of the separated CO₂).

FIG. 1 also includes a small (optional) purge section to recover the CO₂ that exists in the interstitial space between in between sorbent particles or inside a monolith. An additional cooling stage may also be added if needed.

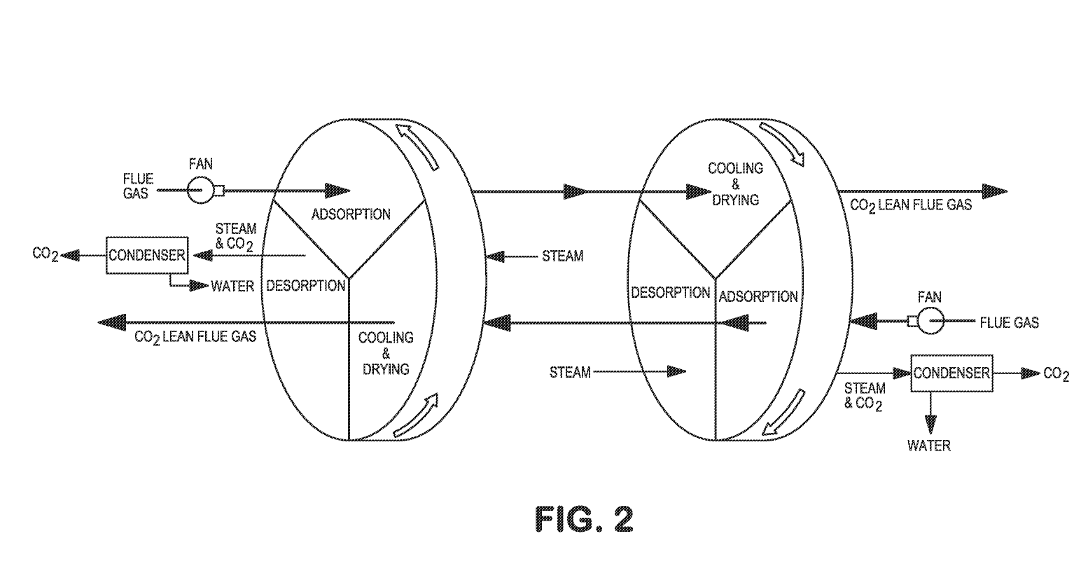

FIG. 2 is a schematic of a two wheel system operated using a method according to the invention, which shows a continuously rotating wheel packed with a CO₂ selective sorbent. The rotating adsorbent can undergo successive steps of (A) CO₂ sorption, (B) CO₂ desorption (e.g., by steam or another fluid), and (C) drying/cooling of the sorbent to the sorption temperature. The cooling step (C) may not be necessary in some cases. For example, no cooling may be needed in situations involving near pressure-based sorption and desorption steps (such as PSA, PPSA, or the like, or combinations thereof). In summary, FIG. 2 depicts a flow coupled process where the CO₂-lean flue gas discharged from one rotating wheel can be used to dry/cool a second sorbent wheel, and vice versa.

It is noted that the discharged CO₂-lean gas can have one or more of the following desirable characteristics that can allow it to be an effective coolant: the discharged CO₂-lean gas can have a relatively large volumetric flow rate that can be as much as about 80 or 90% of the flue gas being processed; and/or the discharged CO₂-lean flue gas can advantageously be low in relative humidity, which can increase the driving force for drying, thereby increasing its effectiveness for drying the sorbent. On the negative side, the CO₂-lean flue gas can generally be somewhat hotter than the flue gas, because of the heat of adsorption. It is estimated that the CO₂-lean flue gas can be up to about 25° C. hotter than the flue gas that is being treated, but the dry gas can advantageously be cooled prior to use in such situations. Of particular interest is the evaporative cooling of the CO₂-lean flue gas prior to its use in cooling the sorbent; it is estimated that up to about 25° C. cooling or more can be achievable by evaporative cooling of the CO₂-lean flue gas.

Multistage (e.g., 2-stage) evaporative cooling may optionally be used to achieve sorbent cooling. In multistage evaporative cooling, the CO₂-lean flue gas can be first cooled using indirect cooling (preferably without adding any moisture). This can be achieved, e.g., by passing the CO₂-lean flue gas inside a heat exchanger externally cooled by evaporative cooling, such as using water spray and a fan.

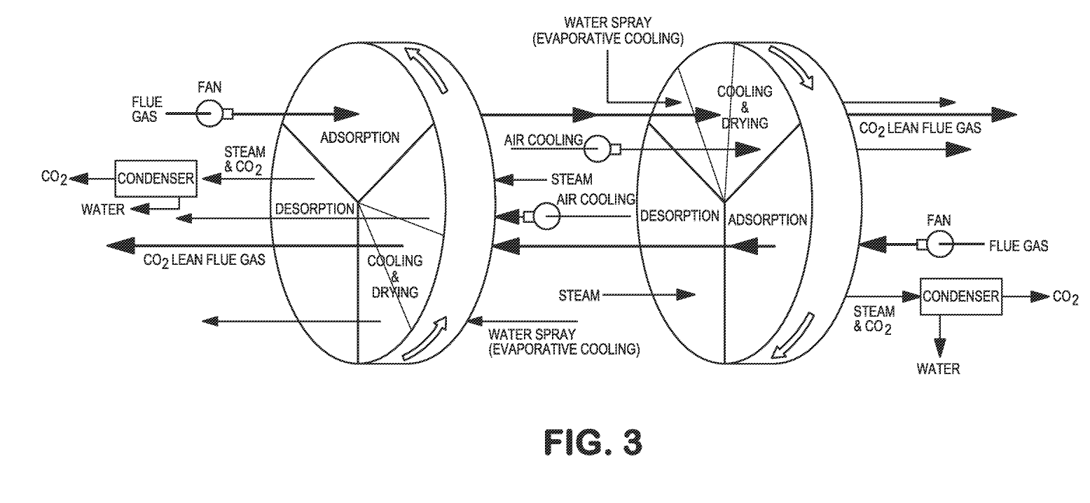

Other cooling fluids can optionally be used instead of or in conjunction with the CO₂-lean gas to achieve sorbent cooling, which can be particularly attractive in embodiments where the CO₂-lean gas, although available in a relatively large volume, may still not be sufficient to achieve sorbent cooling to a desired temperature. FIG. 3 shows such a scenario where water and ambient air can be used in conjunction with the CO₂-lean flue gas to achieve the sorbent cooling.

Indeed, FIG. 3 depicts a scenario where initial adsorbent cooling can be achieved by a water spray, followed by additional drying and cooling with CO₂-lean flue gas and air. The sequence in which the various coolants may be used can be tailored/optimized, based upon their available volumes. An additional blower may be needed if cooling by air is deployed.

In some situations, there may be a concern that moisture in the ambient air can “wet” the sorbent prior to the sorption step. In such situations, an air cooling step can optionally precede the cooling, e.g., using dry CO₂-lean gas.

Co-current cooling can be an alternative to countercurrent cooling. If multiple streams are used for cooling, the coldest steam can advantageously be used the last during the cooling step. This can facilitate the zone where sorption is taking place to stay the coldest, and the migrating hot sorbent zone to stay downstream of the migrating sorption zone. The benefits of this approach can be further enhanced by using a sorbent with a relatively high sorption capacity and a relatively low heat capacity, so that the thermal cooling wave can propagate faster than the adsorption wave.

A design using a sorbent with a relatively high sorption capacity and a relatively low heat capacity can create an environment in which the heat generated from sorption is advantageously not significantly sorbed by the solid sorbent but can be swept away by the flowing gas (where the thermal wave can move faster than the adsorption wave). Thus, the section of the sorbent undergoing sorption can stay relatively cold and can lag behind the thermal front. In many embodiments, the criteria for such a design can be represented by the formula: 3Cp_{S}/2Cp_{B}<q/Y, where Cp_{S} represents the heat capacity of the solid sorbent, Cp_{B} represents the heat capacity of flowing gas [in the same units as Cp_{S}], q represents the molar amount of CO₂ gas sorbed in equilibrium in weight ratio with gas phase of composition Y, and Y represents the molar ratio of sorbate (in the gas phase) to carrier gas. Other advantageous aspects of such a design can include: (A) the substrate on which the sorbent is wash coated itself having a relatively low heat capacity; and (B) the substrate having relatively low thermal conductivity. For example, a ceramic substrate can be preferred over a metal substrate. It can also be desirable to have a thermal barrier between the wash coat and the substrate, e.g., so that the sorption heat can remain in the wash coat to be swept away by the flowing gas.

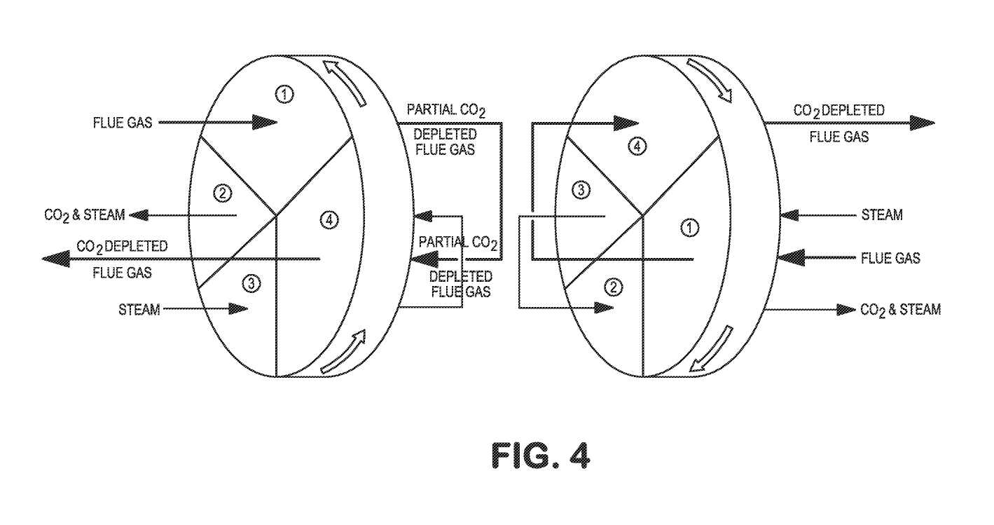

FIG. 4 depicts two rotating wheels, each divided into four sectors, which wheels are pictured as symmetric and mirror images of each other. Each of the two wheels has two sectors reserved for sorption and two sectors reserved for CO₂ steam stripping. If needed, a small cooling sector may optionally be added for cooling the sorbent after the hot stripping steps (not shown). In the configuration of FIG. 4, there is no apparent flow connection between the two wheels—each wheel is represented as being independent of the other wheel. For the rotary wheel on the left, the flue gas can enter a first side of sector (1), and the effluent can be turned around and reenter the opposite side of sector (4) of the wheel. Similarly, the stripping steam can enter a first side of sector (3), and the effluent from sector (3) can be returned to the opposite side of sector (2).

It can be noted that such a flow arrangement can create a counter-current contacting effect within each wheel (similar to described in FIG. 1). In such a flow arrangement, while the flow configuration moves the flue gas from sector (1) to sector (4), the wheel rotation can move the sorbent from sector (4) to sector (1). A similar countercurrent contacting pattern can occur between the stripping sectors (3) and (4). The wheel on the right can advantageously rotate in the opposite direction, but can otherwise have a similar countercurrent contacting pattern. One of the shortcomings of the flow configuration of FIG. 4 is that the flow requires a turn around. This can increase pressure drop and increase plumbing complexity.

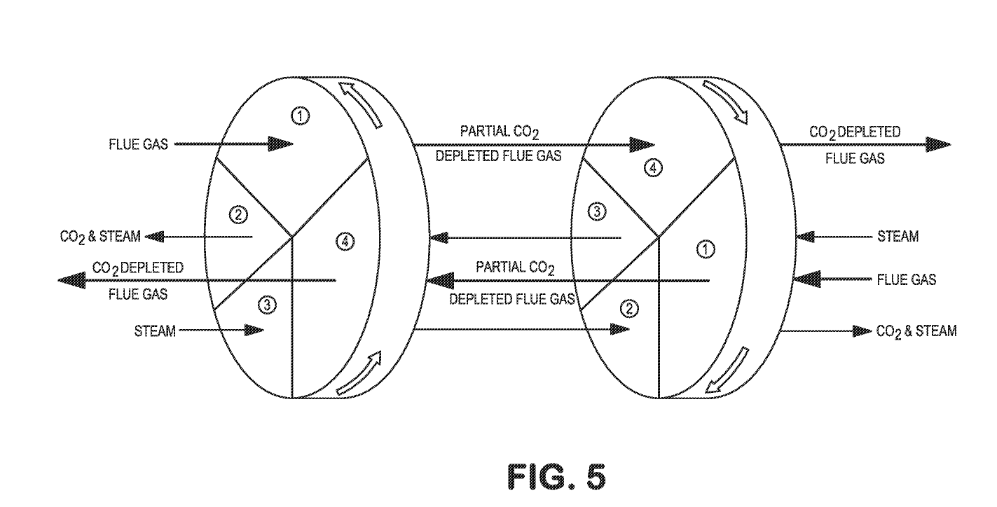

FIG. 5, however, can address some of the shortcomings in the FIG. 4 configuration. FIG. 5 shows integrating (or coupling the flow in) the two wheels, so that a countercurrent contacting pattern can be achieved without the need for any flow turn-around. In FIG. 5, instead of returning the sector (1) effluent to sector (4) in the left wheel, this effluent can be directed to sector (4) of the wheel on the right. Similarly, the effluent stripping steam can be directed to the other wheel, instead of being returned to the same wheel.

It can be convenient to visualize the wheels and flows of FIG. 5 above by laying the flow sheet on a diagram, as shown in FIG. 6. The vertical lines in FIG. 6 represent the solid flow, countercurrently, while the gas flows are represented by horizontal lines. Optionally, instead of the integration of the dual steam stripping between the wheels (partial stripping with an intermediate product from the other wheel before final stripping with fresh steam), the steam stripping in four steps on two wheels can be done with fresh steam in all four steps and with four independent stripped products, to result in substantially no stripping integration between wheels—see FIG. 2, for example. Because such an optional configuration could roughly double the fresh steam requirement for a configuration involving multiple sorption steps on each rotary wheel, it can be less favored than what is shown in FIG. 5.

Additionally or alternately, the present invention can include one or more of the following embodiments.

### Embodiment 1

A method for enhanced control, separation, and/or purification of CO₂ gas from one or more sources having a mixture of gases (and/or carbonaceous liquids having sufficient vapor pressure), the method comprising: providing at least two solid monolithic sorbents having a selectivity for the CO₂ gas in a continuous or semi-continuous, cyclic, countercurrent sorption-desorption process involving at least steps of first and second CO₂ sorption, sorbent heating, first and second CO₂ desorption, and sorbent cooling; in the first CO₂ sorption step, exposing the mixed gas source(s), which contain(s) CO₂ gas at a first temperature, to the solid monolithic sorbents, which are at a second temperature that is at least about 15° C. higher (e.g., at least about 30° C. higher) than the first temperature, as well as under further conditions sufficient for the solid monolithic sorbents to selectively adsorb the desired CO₂ gas, thus forming at least partially, selectively CO₂-sorbed solid monolithic sorbents and an at least partially, selectively CO₂-depleted product stream, and thus simultaneously heating the solid monolithic sorbents to a third temperature higher than the second temperature; P optionally further heating the selectively-CO₂-sorbed solid monolithic sorbent to a fourth temperature above the third temperature in the first CO₂ sorption step, in order to facilitate more efficient desorption; in the first CO₂ desorption step, exposing the CO₂-sorbed and heated solid monolithic sorbents to an at least partially stripped product stream containing desorbed CO₂ and moisture, thus forming at least partially CO₂-desorbed and heated monolithic sorbents, which are further heated to a fifth temperature higher than the third or fourth temperature and which contain moisture, and a further stripped product stream containing additional desorbed CO₂ and a lower moisture content than in the at least partially stripped product stream; in the second CO₂ desorption step, exposing the at least partially CO₂-desorbed and heated solid monolithic sorbents to a CO₂ stripping stream containing moisture and not more than about 1 vol % CO₂ to further desorb CO₂, thus forming further CO₂-desorbed and heated monolithic sorbents, which are further heated to a sixth temperature higher than the fifth temperature and which contain additional moisture, and the at least partially stripped product stream containing desorbed CO₂ and moisture used in the first CO₂ gas desorption step; in the sorbent cooling step, exposing the further CO₂-desorbed and heated monolithic sorbents to a cooling stream at a seventh temperature lower than the second temperature, in order to cool the solid monolithic sorbents to an eighth temperature higher than the seventh temperature; optionally further exposing the monolithic sorbents to a further drying stream to thus form cooled and dried monolithic sorbents having sorbed moisture and a drying throughput stream, at least a portion of which drying throughput stream can optionally be recycled to the source(s) of mixed gas used in the first CO₂ sorption step; in the second CO₂ sorption step, exposing the at least partially, selectively CO₂-depleted product stream from the first CO₂ sorption step to the cooled and optionally dried solid monolithic sorbents under conditions sufficient for the cooled and optionally dried monolithic sorbents to selectively sorb additional CO₂ gas from the at least partially CO₂-depleted product stream, thus forming the at least partially CO₂-sorbed solid monolithic sorbents and a further CO₂-depleted product stream, and thus simultaneously heating the solid monolithic sorbents to the second temperature; and optionally condensing moisture as water from the at least partially stripped product stream and/or from the further stripped product stream, thus forming one or more condensed product streams and thereby decreasing CO₂ concentration in the condensed product stream(s).

### Embodiment 2

The method of embodiment 1, wherein the at least two solid monolithic sorbents are oriented such that their cross-sectional planes are approximately parallel and such that they rotate about a common rotational axis that is substantially perpendicular to the cross-sectional planes of the monolithic sorbents, with each successive solid monolithic sorbent having counter-rotational directions that alternate between clockwise and counterclockwise, as viewed along the common rotational axis.

### Embodiment 3

The method of embodiment 1 or embodiment 2, comprising two solid monolithic sorbents, a first and a second, and thus two sets of streams for each step, also a first and a second, wherein: in the first CO₂ sorption step, the first mixed gas source is exposed to the first solid monolithic sorbent to form the first at least partially CO₂-sorbed solid monolithic sorbent and the first at least partially CO₂-depleted product stream, and the second mixed gas source is exposed to the second solid monolithic sorbent to form the second at least partially CO₂-sorbed solid monolithic sorbent and the second at least partially CO₂-depleted product stream; the first at least partially CO₂-depleted product stream from the first CO₂ sorption step is then exposed to the second cooled and optionally dried monolithic sorbent in the second CO₂ sorption step, thus forming the second further CO₂-depleted product stream, and the second at least partially CO₂-depleted product stream from the first CO₂ sorption step is then exposed to the first cooled and optionally dried monolithic sorbent in the second CO₂ sorption step, thus forming the first further CO₂-depleted product stream; in the second CO₂ desorption step, the first CO₂ stripping stream is exposed to the first at least partially CO₂-desorbed and heated solid monolithic sorbent to form the first further CO₂-desorbed and heated solid monolithic sorbent and the first at least partially stripped product stream, and the second CO₂ stripping stream is exposed to the second at least partially CO₂-desorbed and heated solid monolithic sorbent to form the second further CO₂-desorbed and heated solid monolithic sorbent and the second at least partially stripped product stream; and the first at least partially stripped product stream from the second CO₂ desorption step is then exposed to the second CO₂-sorbed and heated solid monolithic sorbent in the first CO₂ desorption step, thus forming the second further stripped product stream, and the second at least partially stripped product stream from the second CO₂ desorption step is then exposed to the first CO₂-sorbed and heated solid monolithic sorbent in the first CO₂ desorption step, thus forming the first further stripped product stream.

### Embodiment 4

The method of embodiment 1 or embodiment 2, wherein the at least two solid monolithic sorbents each rotate about a rotational axis, and wherein each solid monolithic sorbent is independent of the other(s), such that: in the first CO₂ sorption step, each mixed gas source is exposed to its corresponding solid monolithic sorbent to form its corresponding at least partially CO₂-sorbed solid monolithic sorbent and its corresponding at least partially CO₂-depleted product stream; each at least partially CO₂-depleted product stream from the first CO₂ sorption step is then exposed to its corresponding cooled and optionally dried monolithic sorbent in the second CO₂ sorption step, thus forming its corresponding further CO₂-depleted product stream; in the second CO₂ desorption step, each CO₂ stripping stream is exposed to its corresponding at least partially CO₂-desorbed and heated solid monolithic sorbent to form its corresponding further CO₂-desorbed and heated solid monolithic sorbent and its corresponding at least partially stripped product stream; and each at least partially stripped product stream from the second CO₂ desorption step is then exposed to its corresponding CO₂-sorbed and heated solid monolithic sorbent in the first CO₂ desorption step, thus forming its corresponding further stripped product stream.

### Embodiment 5

The method of any one of the previous embodiments, wherein the solid monolithic sorbents have a CO₂/N₂ selectivity at the operating conditions of at least 4, or alternately of 3 or less.

### Embodiment 6

The method of any one of the previous embodiments, wherein the source(s) of mixed gas each comprise(s) from about 1 vol % to about 25 vol % CO₂ and from about 0.5 vol % to about 20 vol % moisture.

### Embodiment 7

The method of any one of the previous embodiments, wherein the source(s) of mixed gas each comprise(s) from about 10 vol % to about 45 vol % CO₂ and at least about 10 vol % C₁-C₃ hydrocarbons.

### Embodiment 8

The method of any one of the previous embodiments, wherein the source(s) of mixed gas each comprise(s) one or more of the following: from about 5 vppm to about 1000 vppm SOₓ; from about 5 vppm to about 1000 vppm NOₓ; from about 1 vol % to about 40 vol % H₂; from about 10 vppm to about 4000 vppm H₂S; and from about 50 vppm to about 5 vol % CO.

### Embodiment 9

The method of any one of the previous embodiments, wherein the source(s) of mixed gas each comprise(s) a petroleum refinery flue gas stream, a water gas shift process product stream, a hydrocarbon conversion catalyst regeneration gas, a hydrocarbon combustion gas product stream, a virgin or partially treated natural gas stream, or a combination thereof.

### Embodiment 10

The method of any one of the previous embodiments, wherein the at least two solid monolithic sorbents are formed from: an alkalized alumina; an alkalized titania; activated carbon; 13X or 5A molecular sieve; a zeolite having framework structure type AEI, AFT, AFX, ATN, AWW, CHA, DDR, EPI, ESV, FAU, KFI, LEV, LTA, PHI, RHO, SAV, or a combination or intergrowth thereof; a cationic zeolite material; a metal oxide whose metal(s) include(s) an alkali metal, an alkaline earth metal, a transition metal, or a combination thereof; a zeolite imidazolate framework material; a metal organic framework material; or a combination thereof

### Embodiment 11

The method of any one of the previous embodiments, wherein the at least two solid monolithic sorbents are formed from an alkalized alumina and/or wherein there is no optional drying step between the sorbent cooling step and the second CO₂ sorption step.

### Embodiment 12

The method of any one of the previous embodiments, wherein the cyclic sorption-desorption process has an average cycle time from about 1 minute to about 30 minutes.

### Embodiment 13

The method of any one of the previous embodiments, wherein the conditions sufficient for the first and second CO₂ desorption steps include a pressure swing/reduction, a temperature swing/increase, or both.

### Embodiment 14

The method of any one of the previous embodiments, wherein the total pressure conditions in the first and second CO₂ sorption, sorbent heating, first and second CO₂ desorption, and sorbent cooling steps of the sorption-desorption process collectively range from about 0.01 psia (about 0.07 kPaa) to about 150 psia (about 1.0 MPaa).

### Embodiment 15

The method of any one of the previous embodiments, wherein the temperature conditions for all the input streams, output streams, and solid monolithic sorbents in the first and second CO₂ sorption, sorbent heating, first and second CO₂ desorption, and sorbent cooling steps of the sorption-desorption process collectively range from about 35° C. to about 205° C.

While the present invention has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention.

What is claimed is:

1. A method for enhanced control, separation, and/or purification of CO₂ gas from one or more sources having a mixture of gases, the method comprising:
providing at least two solid monolithic sorbents having a selectivity for CO₂ sorption in a continuous or semi-continuous, cyclic, countercurrent sorption-desorption process involving at least steps of first and second CO₂ sorption, sorbent heating, first and second CO₂ desorption, and sorbent cooling;
in the first CO₂ sorption step, exposing the mixed gas source(s), which contain(s) CO₂ gas at a first temperature, to the solid monolithic sorbents, which are at a second temperature that is at least about 15° C. higher than the first temperature, as well as under further conditions sufficient for the solid monolithic sorbents to selectively sorb the desired CO₂ gas, thus forming at least partially, selectively CO₂-sorbed solid monolithic sorbents and an at least partially, selectively CO₂-depleted product stream, and thus simultaneously heating the solid monolithic sorbents to a third temperature that is higher than the second temperature;
optionally further heating the selectively-CO₂-sorbed solid monolithic sorbent to a fourth temperature higher than the third temperature in the first CO₂ sorption step, in order to facilitate more efficient desorption;
in the first CO₂ desorption step, exposing the CO₂-sorbed and heated solid monolithic sorbents to an at least partially stripped product stream containing desorbed CO₂ and moisture, thus forming at least partially CO₂-desorbed and heated monolithic sorbents, which are further heated to a fifth temperature higher than the third or fourth temperature and which contain moisture, and a further stripped product stream containing additional desorbed CO₂ and a lower moisture content than in the at least partially stripped product stream;
in the second CO₂ desorption step, exposing the at least partially CO₂-desorbed and heated solid monolithic sorbents to a CO₂ stripping stream containing moisture and not more than about 1 vol % CO₂ to further desorb CO₂, thus forming further CO₂-desorbed and heated monolithic sorbents, which are further heated to a sixth temperature higher than the fifth temperature and which contains additional moisture, and the at least partially stripped product stream containing desorbed CO₂ and moisture used in the first CO₂ gas desorption step;
in the sorbent cooling step, exposing the further CO₂-desorbed and heated monolithic sorbents to a cooling stream at a seventh temperature lower than the second temperature, in order to cool the solid monolithic sorbents to an eighth temperature higher than the seventh temperature;
optionally further exposing the monolithic sorbents to a further drying stream to thus form cooled and dried monolithic sorbents having sorbed moisture and a drying throughput stream, at least a portion of which drying throughput stream can optionally be recycled to the source(s) of mixed gas used in the first CO₂ sorption step;
in the second CO₂ sorption step, exposing the at least partially, selectively CO₂-depleted product stream from the first CO₂ sorption step to the cooled and optionally dried solid monolithic sorbents under conditions sufficient for the cooled and optionally dried monolithic sorbents to selectively sorb additional CO₂ gas from the at least partially CO₂-depleted product stream, thus forming the at least partially CO₂-sorbed solid monolithic sorbents and a further CO₂-depleted product stream, and thus simultaneously heating the solid monolithic sorbents to the second temperature; and
optionally condensing moisture as water from the at least partially stripped product stream and/or from the further stripped product stream, thus forming one or more condensed product streams and thereby decreasing CO₂ concentration in the condensed product stream(s).

2. The method of claim 1, wherein the at least two solid monolithic sorbents are oriented such that their cross-sectional planes are approximately parallel and such that they rotate about a common rotational axis that is substantially perpendicular to the cross-sectional planes of the monolithic sorbents, with each successive solid monolithic sorbent having counter-rotational directions that alternate between clockwise and counterclockwise, as viewed along the common rotational axis.

3. The method of claim 2, comprising two solid monolithic sorbents, a first and a second, and thus two sets of streams for each step, also a first and a second, wherein:
in the first CO₂ sorption step, the first mixed gas source is exposed to the first solid monolithic sorbent to form the first at least partially CO₂-sorbed solid monolithic sorbent and the first at least partially CO₂-depleted product stream, and the second mixed gas source is exposed to the second solid monolithic sorbent to form the second at least partially CO₂-sorbed solid monolithic sorbent and the second at least partially CO₂-depleted product stream;
the first at least partially CO₂-depleted product stream from the first CO₂ sorption step is then exposed to the second cooled and optionally dried monolithic sorbent in the second CO₂ sorption step, thus forming the second further CO₂-depleted product stream, and the second at least partially CO₂-depleted product stream from the first CO₂ sorption step is then exposed to the first cooled and optionally dried monolithic sorbent in the second CO₂ sorption step, thus forming the first further CO₂-depleted product stream;
in the second CO₂ desorption step, the first CO₂ stripping stream is exposed to the first at least partially CO₂-desorbed and heated solid monolithic sorbent to form the first further CO₂-desorbed and heated solid monolithic sorbent and the first at least partially stripped product stream, and the second CO₂ stripping stream is exposed to the second at least partially CO₂-desorbed and heated solid monolithic sorbent to form the second further CO₂-desorbed and heated solid monolithic sorbent and the second at least partially stripped product stream; and
the first at least partially stripped product stream from the second CO₂ desorption step is then exposed to the second CO₂-sorbed and heated solid monolithic sorbent in the first CO₂ desorption step, thus forming the second further stripped product stream, and the second at least partially stripped product stream from the second CO₂ desorption step is then exposed to the first CO₂-sorbed and heated solid monolithic sorbent in the first CO₂ desorption step, thus forming the first further stripped product stream.

4. The method of claim 1, wherein the at least two solid monolithic sorbents each rotate about a rotational axis, and wherein each solid monolithic sorbent is independent of the other(s), such that:
in the first CO₂ sorption step, each mixed gas source is exposed to its corresponding solid monolithic sorbent to form its corresponding at least partially CO₂-sorbed solid monolithic sorbent and its corresponding at least partially CO₂-depleted product stream;
each at least partially CO₂-depleted product stream from the first CO₂ sorption step is then exposed to its corresponding cooled and optionally dried monolithic sorbent in the second CO₂ sorption step, thus forming its corresponding further CO₂-depleted product stream;
in the second CO₂ desorption step, each CO₂ stripping stream is exposed to its corresponding at least partially CO₂-desorbed and heated solid monolithic sorbent to form its corresponding further CO₂-desorbed and heated solid monolithic sorbent and its corresponding at least partially stripped product stream; and
each at least partially stripped product stream from the second CO₂ desorption step is then exposed to its corresponding CO₂-sorbed and heated solid monolithic sorbent in the first CO₂ desorption step, thus forming its corresponding further stripped product stream.

5. The method of claim 1, wherein the solid monolithic sorbents have a CO₂/N₂ selectivity at the operating conditions of at least 4.

6. The method of claim 1, wherein the solid monolithic sorbents have a CO₂/N₂ selectivity at the operating conditions of 3 or less.

7. The method of claim 1, wherein the source(s) of mixed gas each comprise(s) from about 1 vol % to about 25 vol % CO₂ and from about 0.5 vol % to about 20 vol % moisture.

8. The method of claim 1, wherein the source(s) of mixed gas each comprise(s) from about 10 vol % to about 45 vol % CO₂ and at least about 10 vol % C₁-C₃ hydrocarbons.

9. The method of claim 1, wherein the source(s) of mixed gas each comprise(s) one or more of the following: from about 5 vppm to about 1000 vppm SOₓ; from about 5 vppm to about 1000 vppm NOₓ; from about 1 vol % to about 40 vol % H₂; from about 10 vppm to about 4000 vppm H₂S; and from about 50 vppm to about 5 vol % CO.

10. The method of claim 1, wherein the source(s) of mixed gas each comprise(s) a petroleum refinery flue gas stream, a water gas shift process product stream, a hydrocarbon conversion catalyst regeneration gas, a hydrocarbon combustion gas product stream, a virgin or partially treated natural gas stream, or a combination thereof.

11. The method of claim 1, wherein the at least two solid monolithic sorbents are formed from: an alkalized alumina; an alkalized titania; activated carbon; 13X or 5A molecular sieve; a zeolite having framework structure type AEI, AFT, AFX, ATN, AWW, CHA, DDR, EPI, ESV, FAU, KFI, LEV, LTA, PHI, RHO, SAV, or a combination or intergrowth thereof; a cationic zeolite material; a metal oxide whose metal(s) include(s) an alkali metal, an alkaline earth metal, a transition metal, or a combination thereof; a zeolite imidazolate framework material; a metal organic framework material; or a combination thereof.

12. The method of claim 11, wherein the at least two solid monolithic sorbents are formed from an alkalized alumina and wherein there is no optional drying step between the sorbent cooling step and the second CO₂ sorption step.

13. The method of claim 1, wherein the cyclic sorption-desorption process has an average cycle time from about 1 minute to about 30 minutes.

14. The method of claim 1, wherein the conditions sufficient for the first and second CO₂ desorption steps include a pressure swing/reduction, a temperature swing/increase, or both.

15. The method of claim 1, wherein the second temperature is at least about 30° C. higher than the first temperature.

16. The method of claim 1, wherein the total pressure conditions in the first and second CO₂ sorption, sorbent heating, first and second CO₂ desorption, and sorbent cooling steps of the sorption-desorption process collectively range from about 0.01 psia (about 0.07 kPaa) to about 150 psia (about 1.0 MPaa).

17. The method of claim 1, wherein the temperature conditions for all the input streams, output streams, and solid monolithic sorbents in the first and second CO₂ sorption, sorbent heating, first and second CO₂ desorption, and sorbent cooling steps of the sorption-desorption process collectively range from about 35° C. to about 205° C.
